(19) United States
(12) Patent Application Publication
(10) Pub. No.: US 2021/0196697 A1
(43) Pub. Date: Jul. 1, 2021

(54) COMBINATION THERAPIES FOR TREATING BIPOLAR DISORDER AND ADHD, AND METHODS FOR USING THE SAME

(71) Applicant: PsychNostics, LLC, Baltimore, MD (US)

(72) Inventor: Alagu P. THIRUVENGADAM, Baltimore, MD (US)

(73) Assignee: PsychNostics, LLC, Baltimore, MD (US)

(21) Appl. No.: 16/756,960

(22) PCT Filed: Oct. 24, 2018
(86) PCT No.: PCT/US2018/057277

§ 371 Date: Apr. 17, 2020

Related U.S. Application Data
(60) Provisional application No. 62/581,281, filed on Nov. 3, 2017.

(51) Int. Cl. A61K 31/4458; A61K 31/55; A61P 25/28; G01N 33/50

(52) U.S. Cl. CPC A61K 31/4458; A61K 31/55; G01N 2800/52; G01N 33/502; G01N 2800/305; A61P 25/28

(57) ABSTRACT

The present invention relates to pharmaceutical combinations and compositions, and methods of using the same for treatment of attention deficit hyperactivity disorder (ADHD) and bipolar disorder (BD). The invention relates to combination therapies for the treatment of BD and for ADHD, and methods for treating BD and ADHD using such therapies. The present invention also relates to methods of determining an optimal combination drug treatment therapy for BD and for ADHD, methods of optimizing a combination drug treatment therapy for BD and for ADHD, methods of optimizing dosage of a drug in a combination drug treatment therapy for BD and for ADHD, as well as methods for monitoring the efficacy of a combination therapy for the treatment of BD and for ADHD. The present invention involves analyzing the membrane potential of cells isolated from a BD patient treated with the combination therapy and from an ADHD patient treated with the combination therapy, and calculating a membrane potential ratio therefrom.

### FIELD OF THE INVENTION

The present invention relates to the treatment of Bipolar Disorder (BD) and Attention Deficit Hyperactivity Disorder (ADHD), and more specifically, to combination therapies for the treatment of BD and of ADHD, and methods for treating BD and for treating ADHD using such therapies. The present invention relates to a method for optimizing drug therapy treatment for ADHD and a method of optimizing drug dosage for treatment of ADHD. These methods include optimization of a combination therapy for treatment of ADHD, and optimization of a drug dosage in a combination therapy for treatment of ADHD. The methods of the present invention involve analyzing the membrane potential of cells isolated from a ADHD patient, and calculating a membrane potential ratio therefrom. The present invention further relates to increasing the therapeutic efficacy of a drug therapy treatment for ADHD as well as monitoring the efficacy of a combination therapy for the treatment of ADHD, by analyzing the membrane potential of cells isolated from a ADHD patient treated with the combination therapy, and calculating a membrane potential ratio therefrom. The present invention also relates to a method for optimizing drug therapy treatment for BD and ADHD and a method of optimizing drug dosage for treatment of BD and ADHD. These methods include optimization of a combination therapy for treatment of B), and optimization of a drug dosage in a combination therapy for treatment of BD and ADHD. The methods of the present invention involve analyzing the membrane potential of cells isolated from a BD patient, and calculating a membrane potential ratio therefrom. The present invention further relates to increasing the therapeutic efficacy of a drug therapy treatment for BD as well as monitoring the efficacy of a combination therapy for the treatment of BD, by analyzing the membrane potential of cells isolated from a BD patient treated with the combination therapy, and calculating a membrane potential ratio therefrom.

### BACKGROUND OF THE INVENTION

Bipolar disorder (BD) and attention deficit hyperactive disorder (ADHD) are two of the major mental illnesses difficult to diagnose and to treat. Even though Cade J F J., Lithium salts in the treatment of psychotic excitement, Medical Journal of Australia, 2: 349-352 (1949), discovered the mood stabilizing properties of lithium in BD patients during the mid 1900s, the mechanism of action of lithium in BD is still controversial (Goodwin F K and Jamison K R., Manic-Depressive Illness. Oxford University Press 2007; see also Goodwin F K, Ghaemi N S., The impact of the discovery of lithium on psychiatric thought and practice in the USA and Europe, Australian and New Zealand Journal of Psychiatry, 33: S54-S64 (1999); and also Manji H K, Bowden C L and Belmaker R H. (Ed), Bipolar Medications-Mechanisms of Action, American psychiatric Press, Washington D.C. (2000); also Fieve R R, Lithium Therapy at the Millennium: A Revolutionary Drug Used for 50 Years Faces ompeting Options and Possible Demise, Editorial. Bipolar Disorder, 2: 67-70 (1999)). However Schou M., The early European lithium studies. Australian and New Zealand Journal of Psychiatry, 33: S39-S47 (1999), conducted extensive clinical trials and established lithium's mood stabilizing power in BD patients.

Lithium is the only clinically proven mood stabilizer in BD (Goodwin F K and Jamison K R., Manic-Depressive Illness. Oxford University Press 2007; see also Goodwin F K, Ghaemi N S., The impact of the discovery of lithium on psychiatric thought and practice in the USA and Europe, Australian and New Zealand Journal of Psychiatry, 33: S54-S64 (1999); Manji H K, Bowden C L and Belmaker R H. (Ed)., Bipolar Medications-Mechanisms of Action, American psychiatric Press, Washington D.C. (2000)). Its toxic level is about 2 mM whereas its therapeutic level is around 1.2 mM. The side effects at this level include nausea, diarrhea, dizziness, muscle weakness, fatigue, and a dazed feeling. These unwanted side effects often improve with continued use. Fine tremor, frequent urination, and thirst can occur and may persist with continued use. Weight gain and swelling from excess fluid can also occur. Periodic Blood tests are required. All these symptoms are dosage dependant. Patients' tolerance and compliance at high therapeutic levels are limited.

Hokin L E., Lithium increases accumulation of second messenger inositol 1,4,5-triphosphate in brain cortex slices in species ranging from mouse to monkey, Advanced Enzyme Regul. Pergamon Press Ltd., 33: 299-312 (1993) and his colleagues found that the hydrolysis of the membrane bound phospholipid phosphatidylinositol 4,5-bisphosphate (PIP2) into inositol triphosphate (IP3) and diacylglycerol (DAG) is promoted by lithium in brain cortex slices in species ranging from mouse to monkey. This process is further enhanced by cholinergic agonists such as carbachol. IP3 and DAG play key roles in transmitting the biological signals from the membrane bound G-protein Coupled Receptors (GPCR) to the critical proteins within the cell.

Mental illness afflicts nearly ten percent of the general population both in the United States and in the rest of the world. Bipolar (manic depressive) disorder occurs in one to two percent of the population, and is the sixth leading cause of disability (Coryell et al., Am. J. Psychiatry 150:720-727 (1993); Lopez et al., Nat. Med 4:1241-1243 (1998); Hyman, S. E., Am. J Geriatr. Psychiatry 9:330-339 (2001)). A problem facing the medical community is misdiagnosis of bipolar disorder. Misdiagnosed patients receive an average of 3.5 misdiagnoses and consult four physicians before receiving an accurate diagnosis (“Living with bipolar disorder How far have we really come?” National Depressive and Manic-Depressive Association, Chicago, Ill. (2001)).

Attention-deficit/hyperactivity disorder is characterized by persistent inattention and impulsivity. The criteria for this disorder are outlined, for example, in DSM-IV-TR (Diagnostic and Statistical Manual of Mental Disorders, Fourth Edition, Text Revision 2000, American Psychiatric Association, Washington D.C. (2000)). However misdiagnosis and over-diagnosis are common due to a number of barriers including limited access to available mental health services (National Institute of Health. Diagnosis and treatment of attention deficit hyper activity disorder. (1998); NIH Consensus Statement, 16(2):1-37 Foy J. M. and Earls, M. F., Pediatrics 115:97-104 (2005)).

BD is one of the major mental illnesses difficult to diagnose and to treat. Even though Cade (1) discovered the mood stabilizing properties of lithium in BD patients during the mid 1900s, the mechanism of action of lithium in BD is still controversial (Goodwin and Jamison (2); Manji, Bowden and Belmaker (3), and Fieve (19)). However Schou (4) conducted extensive clinical trials and established lithium's mood stabilizing power in BD patients. Lithium is the only clinically proven mood stabilizer used to treat BD (2, 3). Its toxic level is about 2 mM whereas its therapeutic level is around 1.2 mM. The side effects at this level include nausea, diarrhea, dizziness, muscle weakness, fatigue, and a dazed feeling. These unwanted side effects often improve with continued use. Fine tremor, frequent urination, and thirst can occur and may persist with continued use. Weight gain and swelling from excess fluid can also occur. Periodic Blood tests are required. All these symptoms are dosage dependant. Patients' tolerance and compliance at high therapeutic levels are limited. Lithium is the only clinically-proven mood stabilizer used to treat bipolar disorder. (Goodwin et al., Manic-Depressive Illness, Oxford University Press, 2007; Goodwin et al., “The impact of the discovery of lithium on psychiatric thought and practice in the USA and Europe,” Australian and New Zealand Journal of Psychiatry, 1999, 33: S54-S64; Manji et al., Bipolar Medications-Mechanisms of Action, American psychiatric Press, Washington D.C. 2000). Schou (“The early European lithium studies,” Australian and New Zealand Journal of Psychiatry, 1999, 33: S39-S47) conducted extensive clinical trials and established lithium's mood stabilizing power in BD patients. However, the concentration at which it is generally recognized as being therapeutic (around 1.2 mM) is close to the concentration at which it is toxic (about 2 mM). Thus, since the therapeutic concentration is so close to the concentration at which it is toxic, lithium often causes severe side effects that are not well tolerated by patients. For example, even at the therapeutic concentration of 1.2 mM, side effects may result including nausea, diarrhea, dizziness, muscle weakness, fatigue, and a dazed feeling. Although these unwanted side effects often improve with continued use, fine tremor, frequent urination, and thirst can occur and may persist even with continued use. Weight gain and swelling from excess fluid may also occur from continued use. Because of this battery of side effects, lithium is often poorly tolerated by BD patients, and compliance at high therapeutic levels is limited. Additionally, to balance efficacy with the goal of minimizing side effects, frequent blood tests are required to ensure that the lithium concentration in BD patients remains at a therapeutic, but below toxic, concentration. These side effects, however, are dose-dependent. These findings highlight the persistent and chronic nature of bipolar disorder as well as the magnitude of unmet needs in its treatment.

### SUMMARY OF THE INVENTION

The present invention relates to the fields of clinical psychiatry, clinical psychology and more specifically to the treatment of patients with ADHD using combination therapies. The present invention also relates to determining the optimum dose of a combination therapy for the treatment of ADHD, by analyzing the membrane potential of cells isolated from a ADHD patient treated with the combination therapy, and calculating a membrane potential ratio therefrom.

The present invention further relates to monitoring the efficacy of a combination therapy for the treatment of ADHD by analyzing the membrane potential of cells isolated from a ADHD patient treated with the combination therapy and calculating a membrane potential ratio therefrom.

In one aspect, the present invention provides a method of determining an optimal combination drug treatment therapy for a patient with ADHD, that comprises obtaining a ratio of a mean membrane potential that is a mean membrane potential of a first population of cells from the ADHD patient incubated in vitro in the presence of an agent that alters diacylglycerol signaling and in the absence of K⁺, to a mean membrane potential of a second population of cells from the ADHD patient incubated in vitro in the absence of the test agent that alters diacylglycerol signaling and in the presence of K⁺ or absence of K⁺; comparing the ratio of the mean membrane potential to (a) and/or (b): (a) a control ratio of a mean membrane potential of first population of control human cells known to not have ADHD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+, (b) an ADHD control ratio of a mean membrane potential of first population of ADHD control human cells known to have ADHD incubated in vitro in the presence of the agent that alters diacylglycerol signaling and in the absence of K+, to a mean membrane potential of a second population of the ADHD control human cells incubated in vitro in the absence of the agent that alters diacylglycerol signaling and in the presence of K+ or absence of K+; and identifying the optimal combination drug treatment therapy when the ratio of the mean membrane potential obtained is not significantly different from the control ratio of (a), is decreased towards the control ratio (a) in comparison to or relative to the ADHD control ratio of (b), and/or is decreased in comparison to or relative to the ADHD control ratio of (b).

In a second aspect, the present invention provides a method of optimizing a combination drug treatment therapy for a patient with ADHD, comprising the steps of: obtaining at least one sample from an ADHD patient in a drug therapy treatment for ADHD; performing on each sample, a mean membrane potential test comprising obtaining a ratio of a mean membrane potential that is a mean membrane potential of a first population of cells from the sample incubated in vitro in the presence of an agent that alters diacylglycerol signaling and in the absence of K⁺, to a mean membrane potential of a second population of the sample incubated in vitro in the absence of the test agent that alters diacylglycerol signaling and in the presence of K⁺ or absence of K⁺; comparing the ratio of the mean membrane potential to (a) and/or (b): (a) a control ratio of a mean membrane potential of a first population of control human cells known to not have ADHD incubated in vitro in the presence of the agent that alters diacylglycerol signaling and in the absence of K⁺, to a mean membrane potential of a second population of the control human cells incubated in vitro in the absence of the agent that alters diacylglycerol signaling and in the presence of K+ or absence of K+, (b) an ADHD control ratio of a mean membrane potential of a first population of ADHD control human cells known to have ADHD incubated in vitro in the presence of the agent that alters diacylglycerol signaling and in the absence of K+, to a mean membrane potential of a second population of the ADHD control human cells incubated in vitro in the absence of the agent that alters diacylglycerol signaling and in the presence of K+ or absence of K+; determining an optimal drug therapy treatment for the ADHD patient when the ratio of the mean membrane potential obtained is not significantly different from the control ratio of (a), is decreased towards the control ratio (a) in comparison to or relative to the ADHD control ratio of (b), and/or is decreased in comparison to or relative to the ADHD control ratio of (b). The method may further include optionally, modifying at least one drug in the drug therapy treatment for ADHD when the least one drug treatment therapy for ADHD is detcrmincd to not be the optimal drug treatment therapy for the ADHD patient based on the mean membrane potential. For instance, such as when the ratio of the mean membrane potential obtained is higher in comparison to or relative to the control ratio of (a), is increased towards the ADHD control ratio of (b) in comparison to or relative to the control ratio of (a), and/or is not significantly different from the ADHD control ratio of (b).

In a third aspect, the present invention provides a method for determining an optimum dosage of at least one drug in a combination drug treatment therapy for the treatment of ADHD, said method comprising: obtaining at least one sample from an ADHD patient treated with a dosage of a drug in a combination therapy; performing on each sample, a mean membrane potential test comprising: obtaining a ratio of a mean membrane potential that is a mean membrane potential of a first population of cells from the ADHD patient incubated in vitro in the presence of an agent that alters diacylglycerol signaling and in the absence of K⁺, to a mean membrane potential of a second population of cells from the ADHD patient incubated in vitro in the absence of the test agent that alters diacylglycerol signaling and in the presence of K⁺ or absence of K⁺; comparing the ratio of the mean membrane potential to (a) and/or (b): (a) a control ratio of a mean membrane potential of a first population of cells from a control human known to not have said ADHD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+, (b) an ADHD control ratio of a mean membrane potential of a first population of cells from a control human known to have said ADHD incubated in vitro in the presence of the agent that alters diacylglycerol signaling and in the absence of K+, to a mean membrane potential of a second population of cells from the ADHD control human incubated in vitro in the absence of the agent that alters diacylglycerol signaling and in the presence of K+ or absence of K+; determining the dosage of the at least one drug in the combination drug treatment therapy is an optimal dosage for treating ADHD in the combination therapy when the ratio of the mean membrane potential obtained is not significantly different from the control ratio of (a), is decreased towards the control ratio (a) in comparison to or relative to the ADHD control ratio of (b), and/or is lower in comparison to or relative to the ADHD control ratio of (b), or determining the dosage of the drug in the combination drug treatment therapy is not the optimal dosage for treating ADHD in the combination therapy based on the mean membrane potential. For instance, such as when the ratio of the mean membrane potential obtained is significantly higher in comparison to or relative to the control ratio of (a), is increased towards the ADHD control ratio of (b) in comparison to or relative to the control ratio of (a), and/or is not significantly different from the ADHD control ratio of (b). The method may further include optionally, modifying the dosage of the drug in the combination drug treatment therapy when the dosage of the at least one drug in the combination therapy is determined to be not the optimal dosage for treating ADHD based on the mean membrane potential test.

In a fourth aspect, the present invention provides a method for monitoring the efficacy of a combination drug treatment therapy for the treatment of ADHD, said method comprising: obtaining at least one sample from an ADHD patient treated with a combination drug treatment therapy for treating ADHD; performing on each sample, a mean membrane potential test comprising: obtaining a ratio of a mean membrane potential that is a mean membrane potential of a first population of cells from the ADHD patient incubated in vitro in the presence of an agent that alters diacylglycerol signaling and in the absence of K % to a mean membrane potential of a second population of cells from the ADHD patient incubated in vitro in the absence of the test agent that alters diacylglycerol signaling and in the presence of K⁺ or absence of K; comparing the ratio of the mean membrane potential to (a) and/or (b): (a) a control ratio of a mean membrane potential of a first population of cells from a control human known to not have said ADHD incubated in vitro in the presence of the agent that alters diacylglycerol signaling and in the absence of K+, to a mean membrane potential of a second population of cells from the control human known to not have said ADHD incubated in vitro in the absence of the agent that alters diacylglycerol signaling and in the presence of K+ or absence of K+, (b) an ADHD control ratio of a mean membrane potential of a first population of cells from an ADHD control human known to have said ADHD incubated in vitro in the presence of the agent that alters diacylglycerol signaling and in the absence of K+, to a mean membrane potential of a second population of cells from the ADHD control human incubated in vitro in the absence of the agent that alters diacylglycerol signaling and in the presence of K+ or absence of K+; determining the combination drug treatment therapy is efficacious based on the mean membrane potential test when the ratio of the mean membrane potential obtained is not significantly different from the control ratio of (a), is decreased towards the control ratio (a) in comparison to or relative to the ADHD control ratio of (b), and/or is significantly lower in comparison to or relative to the ADHD control ratio of (b), or determining the combination drug treatment therapy is not efficacious based on the mean membrane potential test. For instance, such as when the ratio of the mean membrane potential obtained is higher in comparison to or relative to the control ratio of (a), is increased towards the ADHD control ratio of (b) in comparison to or relative to the control ratio of (a), and/or is not significantly different from the ADHD control ratio of (b). The method may further include optionally, adjusting a dosage of one or more agents in the combination drug treatment therapy when the combination therapy is determined to be not efficacious based on the mean membrane potential test.

In the methods described herein, the present invention may further include obtaining an initial ratio of a mean membrane potential from an initial population of cells from the human patient before the obtaining step.

The human cells useful in the present methods may be selected from the group consisting of red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells.

In a preferred embodiment, the human cells is selected from the group consisting of red blood cells and lymphoblasts.

The combination drug treatment therapy useful in the present methods is a synergistic combination.

The combination drug treatment therapy may comprise a central nervous system stimulant and at least one adjunctive agent. In particular, the combination drug treatment therapy may comprise methylphenidate and at least one adjunctive agent.

The at least one adjunctive agent useful in the present methods may be an anticholinergic agent such as an antimuscarinic agent or an antinicotinic agent.

The antimuscarinic agent may be selected from the group consisting of trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may be selected from the group consisting of bupropion, dextromethorphan, doxacurium, hexamethonium, mecamylamine, tubocurarine, and pharmaceutically acceptable salts thereof.

The agent that alters diacylglycerol signaling of the present methods may be selected from the group consisting of a calcium-calmodulin (Ca²⁺/CaM) kinase inhibitor, a diacylglycerol kinase inhibitor, a protein kinase C inhibitor, and an agent that affects calcium-activated potassium (CaK) channels. In a preferred embodiment, the agent is a calcium-calmodulin (Ca²⁺/CaM) kinase inhibitor such as autocamtide-2-related inhibitory peptide (AIP). In another preferred embodiment, the agent is a diacylglycerol kinase inhibitor, such as 6-[2-[4-[(4-fluorophenyl)phenylmethylene]-1-piperidinyl]ethyl]-7-methyl-5H-thiazolo[3,2-a]pyrimidin-5-one (ALX).

The mean membrane potential test of the present methods may further include incubating the cells in vitro in buffer comprising a potential-sensitive dye, resuspending the cells in potential-sensitive dye free-buffer, and measuring the cell fluorescence.

The agent that alters K⁺ channel activity of the present methods may be ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

In another aspect, the present invention provides a pharmaceutical combination comprising a central nervous system stimulant and at least one adjunctive agent.

In a further aspect, the present invention provides a pharmaceutical composition comprising methylphenidate or a pharmaceutically acceptable salt thereof, and at least one anticholinergic agent.

In a further aspect, the pharmaceutical composition may further include a pharmaceutically acceptable carrier.

The anticholinergic agent may be an antimuscarinic agent or an antinicotinic agent.

The antimuscarinic agent may be selected from the group consisting of trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may be selected from the group consisting of bupropion, dextromethorphan, doxacurium, hexamethonium, mecamylamine, tubocurarine, and pharmaceutically acceptable salts thereof.

Kits of the present invention are provided comprising (a) a K⁺-containing HEPES reference buffer; (b) a K⁺-free HEPES buffer; and (c) a potential-sensitive dye. The kits further include respectively, instructions for performing an assay to determine an optimal combination drug treatment therapy for ADHD, instructions for performing an assay to optimize a combination drug treatment therapy for ADHD, instructions for performing an assay to determine an optimum dosage of a drug in combination drug treatment therapy for ADHD, and instructions for performing an assay to monitor the efficacy of a combination drug treatment therapy for ADHD.

### BRIEF DESCRIPTION OF THE DRAWINGS

FIG. 1 depicts a comparison of the performance of 1 mM Li, with that of 0.5 mM Li+2.5 μM inositol+10 μM carbachol, using the MPR™ test. The synergistic combination of 0.5 mM lithium with carbachol yielded a higher mean MPR™ value of 0.860, as compared to just 0.814 with 1 mM Li alone.

FIG. 2 depicts a comparison of the performance of 1 mM Li, with that of 0.5 mM Li+2.5 μM inositol+100 ng/ml clozapine, using the MPR™ test. The synergistic combination of 0.5 mM lithium with clozapine yielded a higher mean MPR™ value of 0.804, as compared to just 0.757 with 1 mM Li alone. The minimum therapeutic concentration of clozapine starts at 200 ng/ml of blood serum. This result shows significant improvement at half the concentrations of both lithium and clozapine.

FIG. 3 depicts a comparison of the performance of 1 mM Li, with that of 0.5 mM Li+2.5 μM inositol+10 ng/ml donepezil, using the MPR™ test. The synergistic combination of 0.5 mM lithium with donepezil yielded a higher mean MPR™ value of 0.7%, as compared to just 0.780 with 1 mM Li alone. The minimum therapeutic concentration of donepezil starts at 30 ng/ml of blood serum. This result shows significant improvement at half the concentration of lithium in combination with one third the therapeutic concentration of donepezil.

### DETAILED DESCRIPTION OF THE INVENTION

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article, and “the” and similar referents in the context of describing the invention is to be construed to cover both the singular and the plural, unless otherwise indicated herein or clearly contradicted by context. By way of example, “a human cell” means one human cell or more than one human cell.

The terms “agent(s)”, “modulator(s)”, “test agent(s)”, and “compound(s)” are used herein interchangeably and are meant to include, but are not limited to, peptides, nucleic acids, carbohydrates, small organic molecules, and any other molecules (including, but not limited to, chemicals, metals, and organometallic compounds).

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

In the experiments described herein, the membrane potentials of human cells such as whole blood cells are ascertained and compared. However, the methods of the present invention may use any cell type, such as, but not limited to, erythrocytes, lymphoblasts, platelets, leukocytes, macrophages, monocytes, dendritic cells, fibroblasts, epidermal cells, mucosal tissue cells, cells in the cerebrospinal fluid, and hair cells. Preferably, cells in blood, skin cells, hair cells, or mucosal tissue cells are used because of the ease of harvesting these cell types.

Most biological cells are enclosed by a semi-permeable lipid bilayer which is electrically charged. The electrical voltage across the membrane is called the membrane potential (MP). This potential arises from the ionic gradients between the interior concentrations of ions and the exterior concentration of ions. El Mallakh et al., J. Affect. Disord., 41: 33-37 (1996), measured the MP of white blood cells drawn from the blood of hospitalized bipolar disorder (BD) patients, euthymic patients on lithium and matched non-psychiatric controls. They found that the MP of hospitalized BD patients was hyperpolarized compared to the controls. The MP of cells from euthymic patients was slightly depolarized. Around the same time, Thiruvengadam (Thiruvengadam A. Effect of lithium and sodium valproate ions on resting membrane potentials in neurons: an hypothesis. J. Affect. Disord., 65, 95-99 (2001); and Thiruvengadam, A., 2004. The Recent Studies On The Electrobiochemical Aspects Of Bipolar Disorder. In: Brown, M. R. (Ed.). Focus on Bipolar Disorder Research. Nova Science Publishers, New York, pp. 15-35) independently calculated the effect of lithium on MP using the Goldman-Hodgkin-Katz equation for multiple ions and found that the lithium should depolarize the MP. Thiruvengadam developed a ratiometric assay to measure the ratio of the membrane potential called Membrane Potential Ratio (MPR) using a reference buffer and a test buffer (Thiruvengadam A P. Chandrasekaran K. Evaluating the validity of blood-based membrane potential changes for the identification of bipolar disorder 1. J Affect Disord., 100(1-3):75-82 (2007)). The MPR technology is the subject several earlier patents which are also herein incorporated in entirety. The reference buffer contained NaCl, CaCl₂ and glucose at physiological concentrations. The buffering agent hepes was also added to the buffer to maintain the pH. The test buffer contained 30% of ethyl alcohol in addition to the chemicals contained in the reference buffer. The membrane potentials were measured in both the buffers and the ratio of the MIP in the test buffer to the MP in the reference buffer was designated Membrane Potential Ratio (MPR). This ratio was used in all of our clinical trials using patients' whole blood samples.

The first clinical trial was carried out at the University Of Maryland School Of Medicine with a grant from the Technology Development Corporation of Maryland. Hospitalized patients were interviewed by the attending psychiatric department faculty and staff and blood was drawn after their diagnostic evaluation. The final validation was made by the attending faculty using the treatment response of the patients. The first trial involved hospitalized patients and did not include children and adolescents (Thiruvengadam A P. Chandrasekaran K. Evaluating the validity of blood-based membrane potential changes for the identification of bipolar disorder 1. J Affect Disord., 100(1-3):75-82 (2007)). In order to cover a broader range of patient population a second clinical trial was carried out with the participation of several clinical psychiatrists serving the community. The significant result that came out of these clinical trials is that the bipolar group and the ADHD group are significantly different from each other in terms of the MPR values.

MPR Response to Lithium

During the course of these trials, Thiruvengadam and Woodruff discovered that the MPR responds to successful treatment of both BD and ADHD patients with appropriate medications (U.S. application Ser. No. 14/888,720, the disclosure of which is herein incorporated by reference in its entirety). It was shown that the MPR responds to lithium treatment in BD patients and serves as a validation of the MPR test.

DAG Signaling Pathway

In one of the biological signaling pathways, diacylglycerol (DAG) functions as a second messenger signaling lipid. DAG is a product of the hydrolysis of the phospholipid PIP2 (phosphatidyl inositol-bisphosphate) by the enzyme phospholipase C (PLC, a membrane-bound enzyme). It produces inositol trisphosphate (IP₃) through the same reaction. Although inositol trisphosphate (IP₃) diffuses into the cytosol, diacylglycerol (DAG) remains within the plasma membrane due to its hydrophobic properties. The production of DAG in the membrane facilitates translocation of PKC from the cytosol to the plasma membrane (Newton 11). Hence, both DAG and PKC enzyme play important roles in several signal transduction cascades (12). Thiruvengadam (U.S. Pat. No. 7,906,300 B2 pending as of Jun. 5, 2016) identified the modulators of the MPRs of patients' cells that could serve as the drug targets for increasing the MPR values in BD patients to the level of the MPR values of Negatives. This invention further identifies the DAG signaling pathway as the principal signaling mechanism that modulates the MPR values. Furthermore this invention identifies the principal compounds and polypeptides along this pathway as potential diagnostic markers and drug targets for 13D and ADHD. They include DAG and its associated enzymes and kinases, PKC isoforms and associated enzymes and kinases, and Ca²⁺/CaM and its associated enzymes and kinases.

Lithium Increases IP3 and DAG

Hokin (Hokin L E. Lithium increases accumulation of second messenger inositol 1,4,5-triphosphate in brain cortex slices in species ranging from mouse to monkey. Advanced Enzyme Regul. Pergamon Press Ltd., 33: 299-312 (1993)) and his colleagues showed that lithium, at concentrations as low as 1 mM (which is a therapeutic plasma concentration in the treatment of bipolar disorder), increased the accumulation of inositol trisphosphate (IP3) in slices of cerebral cortex of guinea pig, rabbit and monkey (a therapeutically relevant model for humans). Since DAG is another product of the same reaction they presumed that DAG also increased correspondingly. Furthermore, Dixon and Hokin also found that carbachol increased the accumulation of IP3 (Dixon J F, Hokin L E, Kinetic analysis of the formation of inositol 1,2-cyclic phosphate in carbachol-stimulated pancreatic minilobules. Half is formed by direct phosphodiesteratic cleavage of phosphatidylinositol, J. Biol. Chem., 264,11721-11724 (1989)). Berridge and Irvine (Berridge M J, Irvine, Inositol phosphates and cell signalling, Nature 341, 197-205 (1989)) summarized the key reaction of this transducing mechanisms as the hydrolysis of the phosphoinositides to give two products (diacylglycerol and inositol trisphosphate), both of which may function as second messengers to initiate the signaling cascade. Stubbs and Agranoff (Stubbs et al., Lithium Enhances Receptor-Stimulated CDP-Diacylglycerol Formation in Inositol-Depleted SK-N-SH Neuroblastoma Cells, J. Neurochem., 60(4): 1292-1299 (1993)) found that the addition of carbachol to [3H]cytidine-prelabeled cells elicited a four to fivefold increase in the accumulation of labeled CDP-DAG. Hokin (Hokin L E. Lithium increases accumulation of second messenger inositol 1,4,5-triphosphate in brain cortex slices in species ranging from mouse to monkey. Advanced Enzyme Regul. Pergamon Press Ltd., 33: 299-312 (1993)) summarized all these results in his review and concluded that lithium and cholinergic agonists such as carbachol increased IP3 and DAG substantially.

As previously discussed, lithium is the only clinically proven mood stabilizer that works for BD (Goodwin F K and Jamison K R. Manic-Depressive Illness. Oxford University Press 2007. See also Goodwin F K, Ghaemi N S. The impact of the discovery of lithium on psychiatric thought and practice in the USA and Europe. Australian and New Zealand Journal of Psychiatry, 33: S54-S64 (1999)). Its toxic level is about 2 mM whereas its therapeutic level is around 1.2 mM. The side effects at this level includes nausea, diarrhea, dizziness, muscle weakness, fatigue, and a dazed feeling. These unwanted side effects often improve with continued use. Fine tremor, frequent urination, and thirst can occur and may persist with continued use. Weight gain and swelling from excess fluid can also occur. Periodic Blood tests are required. All these symptoms are dosage dependant. Patients' tolerance at high therapeutic levels is limited. The present invention addresses whether the required lithium level may be reduced by a synergic combination of drugs.

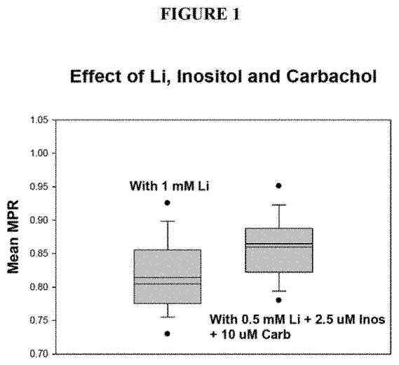

Carbachol is a choline carbamate and is classified as a cholinergic agonist. It is primarily used as an ophthalmic solution for various ophthalmic purposes, such as for treating glaucoma, or for use during ophthalmic surgery. Hokin and his colleagues used it along with lithium and inositol to promote PIP2 hydrolysis (Dixon J F, Hokin L E, Kinetic analysis of the formation of inositol 1,2-cyclic phosphate in carbachol-stimulated pancreatic minilobules. Half is formed by direct phosphodiesteratic cleavage of phosphatidylinositol, J. Biol. Chem., 264.11721-11724 (1989)). Thiruvengadam conducted experiments with carbachol using the MPR test assay (U.S. Pat. No. 7,425,410 B2 and U.S. application Ser. No. 14/888,720; the disclosures of which are herein incorporated by reference in their entirety). MPR™ is the ratio between the MP in the test buffer and that in the reference buffer. The reference buffer contains NaCl, CaCl₂, glucose and Hepes where as the test buffer contains ethyl alcohol (EtOH) in addition to these compounds. Lithium, inositol and carbachol were added to the test buffer in these experiments. Patient's whole blood samples are suspended in both buffers for 20 minutes, spun for five minutes, drained and resuspended in their respective buffers. These samples are distributed in 96 well plates and tested in a plate reader (FLx 800 manufactured by BioTek). As shown in FIG. 1, the MPR value for 1 mM Li was 0.814. The MPR improved to 0.860 with 0.5 mM Li+2.5 uM inositol+10 uM carbachol. This result shows that the MPR value with 1 mM Li can be exceeded with less than 0.5 mM Li in combination with carbachol demonstrating the therapeutic advantages of the combination drug.

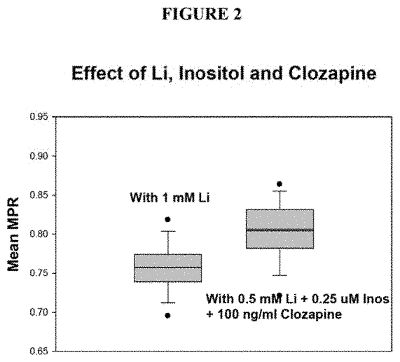

Clozapine is a dibenzodiazepine discovered in the 1960s and used in mental healthcare. It is a cholinergic agonist. It was the first atypical antipsychotic. It is on the World Health Organization's List of Essential Medicines, the most important medications needed in a basic health system. It has been used to treat BD (Calabrese J R, Gajwani P Lamotrigine and Clozapine for Bipolar Disorder, Letter to the Editor, Am J Psychiatry 157:9, page 1523 (2000); see also Calabrese J R, Kimmel S E, Woyshville M J, Rapport D J, Faust C J, Thompson P A, Meltzer H Y: Clozapine for treatment-refractory mania, Am J Psychiatry, 153:759-764 (1996); Frye M A, Ketter T A, Altshuler L L, Denicoff K D, Dunn R T, Kimbrell T A, Cora-Locatelli G, Post R M. Clozapine in Bipolar Disorder: Treatment Implications for Atypical Antipsychotics, J Affec. Disord., 48: 91-104 (1998); see also Vangala V R, Brown E S, Suppes T. Clozapine Associated with Decreased Suicidality in Bipolar Disorder: A Case Report. Bipolar Disord., 2: 123-124 (1999); and Kaplan H I, Sadock B J. Synopsis of Psychiatry. 8th Ed. Baltimore: Williams & Wilkins 1988:103-104). Again the side effects of clozapine are very high at the therapeutic levels ranging upwards of 200 ng/ml of blood plasma. The MPR test was used to evaluate clozapine using a different patient blood sample as a possible synergic compound to reduce the required level of lithium as shown in FIG. 2. The mean MPR value for 1 mM Li was 0.757. The mean MPR improved to 0.804 with 0.5 mM Li+2.5 uM inositol+100 ng/ml clozapine. The therapeutic dosing varies from 200 ng/ml of serum to 1000 ng/ml (17). This result again shows that the MPR value with 1 mM Li can be achieved with less than 0.5 mM Lithium in combination with clozapine at half the minimum therapeutic level currently recommended. This greatly reduces the side effects.

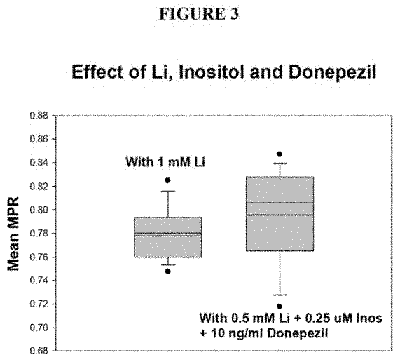

Donepezil is used to improve cognition and behavior of people with Alzheimers disorder. Donepezil is a centrally acting reversible acetylcholinesterase inhibitor. The therapeutic reference range is 30-75 ng/ml (Hefner G, Brueckner A, Geschke K, C Hiemke C, Fellgiebel A, Therapeutic Drug Monitoring (TDM) of donepezil in patients with Alzheimers dementia, Pharmacopsychiatry, 46. A42 (2013)). The MPR test was used to evaluate its synergic effect combined with lithium as shown in FIG. 3. With the addition of 1 mM lithium, the mean MPR is 0.780 for this blood sample. It improved to 0.796 with a combination 0.5 mM Li+2.5 uM inositol+10 ng/ml of donepezil. This result shows that the MPR value with 1 mM lithium can be achieved with 0.5 mM lithium in combination with 10 ng/ml donepezil which is one third of minimum therapeutic dosage currently used. Such a combination greatly reduces the side effects.

PiP2 Hydrolysis and DAG Signaling

The membrane bound phospholipid phosphatidylinositol bisphosphate (PIP2) is a component of the plasma membrane, localized to the inner layer of the phospholipid bilayer. The hydrolysis of PIP2 by phospholipase C (PLC) produces two distinct second messengers, diacylglycerol (DAG) and inositol trisphosphate (P3). Diacylglycerol and IP3 stimulate distinct downstream signaling pathways (protein kinase C and Ca2+ mobilization by calmodulin). The diacylglycerol produced by hydrolysis of PIP2 activates protein-serine/threonine kinases belonging to the protein kinase C family, many of which play important roles in DAG signaling. Although inositol trisphosphate (IP₃) diffuses into the cytosol, diacylglycerol (DAG) remains within the plasma membrane due to its hydrophobic properties. The production of DAG in the membrane facilitates translocation of PKC from the cytosol to the plasma membrane (Newton 15). The PKC activates the calmodulin which in turn modulates the process and transmit this signal to the potassium channels in the cell membrane. Calcium activated potassium channels (CAK channels of which hSK₄ is a member) are activated by Calmodulin (Fanger C M. et al. Calmodulin mediates calcium-dependent activation of the intermediate conductance KCa channel, IKCa1. J. Biol. Chem., 274: 5746-54 (1999)). Calmodulin, CaM, (also called Ca²⁺/CaM) is a widespread and abundant transducer of calcium signaling in cells (Stevens F C, “Calmodulin: an introduction”. Can. J. Biochem. Cell Biol. 61 (8): 906-10 (1983)). It can bind to and regulate a number of different protein targets, thereby affecting many different cellular functions. In the small conductance calcium activated potassium channels (CAK channels), calcium gating is the primary mechanism controlling the potassium flow through the pores. CaM is responsible for this calcium gating (Fanger C M. et al. Calmodulin mediates calcium-dependent activation of the intermediate conductance KCa channel, IKCa1. J. Biol. Chem., 274: 5746-54 (1999)). The synergic combination of lithium with cholinergic agonists promotes the PIP2 hydrolysis and DAG signaling activity as demonstrated by our experiments discussed above.

Synergetic Combination of Drug for ADHD

Just as lithium depolarizes the membrane, methylphenidate hyperpolarizes the membrane as discussed in U.S. application Ser. No. 14/888,720, the disclosure of which is herein incorporated by reference in its entirety. Furthermore, as was shown in U.S. Pat. No. 9,523,673 B2, the disclosure of which is herein incorporated by reference in its entirety, the signaling pathway controls the MPR. As explained above, cholinergic agonists increase the formation of DAG and anticholinergic agents decrease the formation DAG (Kaplan H I, Sadock B J. Synopsis of Psychiatry. 8th Ed. Baltimore: Williams & Wilkins, pages 103-104 (1988)). Therefore, a synergic combination of MPH with an anticholinergic agent enhances the effect of MPH thereby reducing the dosage needed for efficacy in ADHD. That is, the synergistic combination increases the efficacy of MPH for the treatment of ADHD.

Methylphenidate (MPH) Side Effects and Potential for Addiction

MPH is a commonly used drug for the treatment of ADHD. MPH recommended dose is 10-60 mg daily given in 2 or 3 divided doses. Serious side effects may include stomach pain, nausea, vomiting, loss of appetite, vision problems, dizziness, mild headache, sweating, mild skin rash, numbness, tingling, or cold feeling in your hands or feet, nervous feeling, sleep problems (insomnia), and weight loss. MPH can be very addictive, especially when misused or taken via alternate methods, such as by injection or snorting. The Drug Enforcement Administration (DEA) has classified MPH as a Schedule I I drug, meaning it has a high potential for abuse.

The MPR in ADHD patients is generally high as compared to control patients. This is due to the response of the DAG signaling pathway discussed above. MPH reduces the MPR in a dose dependant manner (U.S. application Ser. No. 14/888,720). The second messengers for MPH are inositol triphosphate/diacylglycerol (IP3/DG) via phospholipase C and phospholipid phosphatidylinositol bisphosphate (PIP2) (21). The DAG activity is reduced by MPH by reducing the cleavage of phospholipid phosphatidylinositol bisphosphate (PIP2). Just as cholinergic agents increase PIP2 cleavage, anticholinergic agents decrease the cleaving of PIP2 thereby decreasing the availability of DAG (22, 23). Decreased level of DAG would decrease MPR thereby increasing the efficacy of MPH at low dosages without the deleterious side effects and addiction possibilities. Thus, the combination of MPH with an anticholinergic agent would reduce the required dosage of MPH needed for the effective treatment of ADHD. There are more than 100 potential candidates for using as anticholinergic agents (ACB_scale, Aging Brain Care, agingbraincare.org (2012), and see also “Drugs with Anticholinergic Activity” PL Detail-Document #271206, PHARMACIST'S LETTER/PRESCRIBER'S LETTER, December 2011; the disclosures of which are herein incorporated by reference in their entirety).

Membrane Potential Ratio (MPR™) Differences in BD and ADHD. The mean values of membrane potential ratio (MPR™) for the BD patients are significantly lower than that for the Negatives (who are neither BD nor ADHD). Similarly the membrane potential ratio (MPR™) values for the ADHD patients are significantly higher than that for the negatives.

Most biological cells are enclosed by a semi-permeable lipid bilayer which is electrically charged. The electrical voltage across the membrane is called the membrane potential (MP). This potential arises from the ionic gradients between the interior concentrations of ions and the exterior concentration of ions. El Mallakh et al., Leukocyte transmembrane potential in bipolar illness., J. Affect. Disord., 41: 33-37 (1996), measured the MP of white blood cells drawn from the blood of hospitalized bipolar disorder (BD) patients, euthymic patients on lithium and matched non-psychiatric controls. They found that the MP of hospitalized BD patients was hyperpolarized compared to the controls. The MP of cells from euthymic patients was slightly depolarized. Around the same time. Thiruvengadam (Thiruvengadam A., Effect of lithium and sodium valproate ions on resting membrane potentials in neurons: an hypothesis., J. Affect. Disord., 65, 95-99 (2001); and Thiruvengadam, A., The Recent Studies On The Electrobiochemical Aspects Of Bipolar Disorder. In: Brown, M. R. (Ed.), Focus on Bipolar Disorder Research. Nova Science Publishers, New York, pp. 15-35 (2004); the disclosures of which are herein incorporated by reference in their entirety), independently calculated the effect of lithium on MP using the Goldman-Hodgkin-Katz equation for multiple ions and found that the lithium should depolarize the MP. Thiruvengadam developed a ratiometric assay to measure the ratio of the membrane potential called Membrane Potential Ratio (MPR) using a reference buffer and a test buffer (Thiruvengadam A P. Chandrasekaran K). Evaluating the validity of blood-based membrane potential changes for the identification of bipolar disorder 1. J Affect Disord., 100(1-3): 75-82 (2007); the disclosure of which is herein incorporated by reference in its entirety).

The ratiometric assay to measure the ratio of the membrane potential (MPR) has been described in U.S. Pat. No. 7,425,410, U.S. Pat. No. 906,300, and U.S. patent application Ser. No. 14/888,720, the disclosures of which are herein incorporated by reference in their entirety. The reference buffer contained NaCl, CaCl₂ and glucose at physiological concentrations. The buffering agent hepes was also added to the buffer to maintain the pH. The test buffer contained 30% of ethyl alcohol in addition to the chemicals contained in the reference buffer. The membrane potentials were measured in both the buffers and the ratio of the MP in the test buffer to the MP in the reference buffer was designated Membrane Potential Ratio (MPR). This ratio was used in clinical trials using patients' whole blood samples. The membrane potential ratio (MPR™) values for BD patients were found to be significantly lower than that for negatives (including normals, unipolar depressives, and schizophrenics). On the other hand, the membrane potential ratio (MPR™) values for ADHD patients were found to be significantly higher than that for negatives.

Methods for diagnosing and identifying modulators of membrane potentials in BD and ADHD using membrane potential ratio (MPR™) has been described in U.S. Pat. No. 9,523,673, the disclosure of which is herein incorporated by reference in its entirety.

“Significantly higher”, “significantly lower” or “significantly different” means a value that is considered significant as determined by the various statistical tests and analyses commonly used and known in the art. Membrane potential ratio (MPR™) is the ratio between the membrane potential in the test buffer and that in the reference buffer. The reference buffer contains NaCl, CaCl₂, glucose and Hepes whereas the test buffer contains ethyl alcohol (EtOH) in addition to these compounds. Both buffers do not contain K⁺ ions. The role of the absence of K ions in the buffer on membrane potential and the addition of EtOH needs to be understood in order to explain their effects.

The present invention relates to the fields of clinical psychiatry, clinical psychology and more specifically to optimizing treatment of patients with BD and ADHD using the MPR™ Test after the diagnosis has been made. In particular, the present invention further relates to the synergic combination of lithium and cholinergic agonists in the presence of inositol for BD, and synergic combination of methylphenidate and anticholinergics for ADHD.

The present invention relates to the treatment of ADHD, and more specifically, to combination therapies for the treatment of ADHD, and methods for treating BD and methods for treating ADHD using such therapies.

The present invention relates to the treatment of Bipolar Disorder (BD), and more specifically, to combination therapies for the treatment of BD, and methods for treating BD using such therapies.

The present invention also relates to determining the optimum dose of a combination therapy for the treatment of BD, by analyzing the membrane potential of cells isolated from a BD patient treated with the combination therapy, and calculating a membrane potential ratio therefrom. The present invention further relates to monitoring the efficacy of a combination therapy for the treatment of BD, by analyzing the membrane potential of cells isolated from a BD patient treated with the combination therapy, and calculating a membrane potential ratio therefrom.

The present invention also relates to determining the optimum dose of a combination therapy for the treatment of ADHD, by analyzing the membrane potential of cells isolated from a ADHD patient treated with the combination therapy, and calculating a membrane potential ratio therefrom. The present invention further relates to monitoring the efficacy of a combination therapy for the treatment of ADHD by analyzing the membrane potential of cells isolated from a ADHD patient treated with the combination therapy, and calculating a membrane potential ratio therefrom.

In some aspects, the present invention relates to combination therapies for the treatment of BD. In preferred embodiments thereof, the combination therapy contains lithium and at least one cholinergic agonist.

In some aspects, the present invention relates to combination therapies for the treatment of BD. In preferred embodiments thereof, the combination therapy contains a CNS stimulant and at least one cholinergic agonist. The CNS stimulant is preferably, an amphetamine, and more preferably, methylphenidate.

As noted above, most biological cells are enclosed by a semi-permeable lipid bilayer that is electrically charged. The electrical voltage across the membrane is called the membrane potential (MP). This potential arises from the ionic gradients between the interior concentrations of ions and the exterior concentration of ions. El Mallakh et al. (“Leukocyte transmembrane potential in bipolar illness,” J. Affect. Disord., 1996, 41: 33-37; the disclosure of which is incorporated herein by reference in its entirety) measured the MP of white blood cells drawn from the blood of hospitalized BD patients, euthymic patients on lithium, and matched non-psychiatric controls. They found that the MP of hospitalized RD patients was hyperpolarized compared to the controls. The MP of cells from euthymic patients was slightly depolarized. Around the same time, the present inventor independently calculated the effect of lithium on MP using the Goldman-Hodgkin-Katz equation for multiple ions, and found that lithium should depolarize the MP. Thiruvengadam (“Effect of lithium and sodium valproate ions on resting membrane potentials in neurons: an hypothesis,” J. Affect. Disord., 2001, 65: 95-99; the disclosure of which is incorporated herein by reference in its entirety) and Thiruvengadam (The Recent Studies On The Electrobiochemical Aspects Of Bipolar Disorder. In: Brown, M. R. (Ed.), Focus on Bipolar Disorder Research. Nova Science Publishers, New York, 2004, pp. 15-35; the disclosure of which is incorporated herein by reference in its entirety).

The present inventor developed a ratiometric assay to measure the ratio of the membrane potential called Membrane Potential Ratio (MPR™), using a reference buffer and a test buffer. Thiruvengadam et al. (“Evaluating the validity of blood-based membrane potential changes for the identification of bipolar disorder,” 1. J. Affect Disord., 2007, 100(1-3-75-82, the disclosure of which is incorporated herein by reference in its entirety). The reference buffer may contain NaCl, CaCl₂) and glucose at physiological concentrations. The buffering agent HEPES was also added to the buffer to maintain the pH. The test buffer may contain ethyl alcohol, preferably, 30% of ethyl alcohol, in addition to the chemicals contained in the reference buffer. The test buffer may contain K⁺ or no K⁺. The membrane potentials were measured in both the buffers and the ratio of the MP in the test buffer to the MP in the reference buffer was designated the “Membrane Potential Ratio” (MPR™). Preferably, both the test buffer and the reference buffer contains no K.

The first clinical trial using MPR™ was carried out at the University Of Maryland School Of Medicine. Hospitalized patients were interviewed by the attending psychiatric department faculty and staff and blood was drawn after their diagnostic evaluation. The final validation was made by the attending faculty using the treatment response of the patients. Thiruvengadam et al. (“Evaluating the validity of blood-based membrane potential changes for the identification of bipolar disorder,” I. J. Affect Disord., 2007, 100(1-3): 75-82, the disclosure of which is incorporated herein by reference in its entirety). In order to cover a broader patient population, a second clinical trial was carried out with the participation of several clinical psychiatrists serving the community. These clinical trials showed that the bipolar group and the ADHD group are significantly different from each other in terms of their MPR™ values.

In the present invention, it was found that the MPR™ responds to lithium treatment in BD patients and may serve as a validation of the MPR™ test.

In a biological signaling pathway relevant to MPR™, diacyglycerol (DAG) functions as a second messenger signaling lipid. DAG is a product of the hydrolysis of the phospholipid PIP2 (phosphatidyl inositol-bisphosphate) by the enzyme phospholipase C (PLC, a membrane-bound enzyme). It produces inositol trisphosphate (IP3) through the same reaction. Although inositol trisphosphate (IP3) diffuses into the cytosol, diacylglycerol (DAG) remains within the plasma membrane due to its hydrophobic properties. The production of DAG in the membrane facilitates translocation of PKC from the cytosol to the plasma membrane. Newton (“Protein Kinase C: Poised to signal,” Am. J. Physiol. Endocrinol. Metab., 2010.298:E395-E402). Hence, both DAG and PKC enzyme play important roles in several signal transduction cascades. Nishizuka Y (“The role of protein kinase C in cell surface signal transduction and tumour promotion,” Nature, 1984, 308(5961); 693-8). Thiruvengadam identified the modulators of the MPRs of patients' cells that could serve as the drug targets for increasing the MPR values in BD patients to the level of the MPR values of Negatives, and identified the DAG signaling pathway as a signaling mechanism that modulates the MPR values.

Phosphatidylinositol 4,5-bisphosphate (PIP2) hydrolysis and diacylglycerol (DAG) signaling are coupled together in producing the therapeutic effects of lithium. Hokin showed that lithium, at concentrations as low as 1 mM (which is a therapeutic plasma concentration for the treatment of bipolar disorder), increased the accumulation of inositol trisphosphate (IP3) in slices of cerebral cortex of guinea pig, rabbit and monkey (a therapeutically relevant model for humans). Hokin L E (“Lithium increases accumulation of second messenger inositol 1,4,5-triphosphate in brain cortex slices in species ranging from mouse to monkey,” Advanced Enzyme Regul., 1993, 33; 299-312). Since DAG is another product of the same reaction they presumed that DAG also increased correspondingly. The effect of increases in IP3 and DAG on membrane potential and excitability, and its relevance to MPR™, has previously been discussed. See U.S. Pat. No. 7,906,300.

The membrane bound phospholipid phosphatidylinositol bisphosphate (PIP2) is a component of the plasma membrane, localized to the inner layer of the phospholipid bilayer. The hydrolysis of PIP2 by phospholipase C (PLC) produces two distinct second messengers, diacylglycerol (DAG) and inositol trisphosphate (IP3). Diacylglycerol and IP3 stimulate distinct downstream signaling pathways (protein kinase C and Ca2+ mobilization by calmodulin). The diacylglycerol produced by hydrolysis of PIP2 activates protein-serine/threonine kinases belonging to the protein kinase C family, many of which play important roles in DAG signaling. Although inositol trisphosphate (IP₃) diffuses into the cytosol, diacylglycerol (DAG) remains within the plasma membrane due to its hydrophobic properties. The production of DAG in the membrane facilitates translocation of PKC from the cytosol to the plasma membrane (Newton, A. C., Protein Kinase C: Poised to signal, Am J Physiol Endocrinol Metab., 298: E395-E402, 2010)). The PKC activates the calmodulin which in turn modulates the process and transmit this signal to the potassium channels in the cell membrane. Calcium activated potassium channels (CAK channels of which hSK₄ is a member) are activated by Calmodulin (Fanger C M. et al. Calmodulin mediates calcium-dependent activation of the intermediate conductance KCa channel, IKCa1. J. Biol. Chem., 274: 5746-54 (1999)). Calmodulin, CaM, (also called Ca²⁺/CaM) is a widespread and abundant transducer of calcium signaling in cells (Stevens F C, “Calmodulin: an introduction”. Can. J. Biochem. Cell Biol. 61 (8): 906-10 (1983)). It can bind to and regulate a number of different protein targets, thereby affecting many different cellular functions. In the small conductance calcium activated potassium channels (CAK channels), calcium gating is the primary mechanism controlling the potassium flow through the pores. CaM is responsible for this calcium gating (12). The synergic combination of lithium with cholinergic agonists promotes the PIP2 hydrolysis and DAG signaling activity as demonstrated herein.

Recent clinical trials using human whole blood samples have shown that that the Membrane Potential Ratios (MPR™) are significantly different among Bipolar Disorder (BD) patients, Attention Deficit Hyperactive Disorder (ADHD) patients and the negative group who are neither BD nor ADHD. These experiments involve a test buffer with no K+ ions, but it contains ethyl alcohol (EtOH). The membrane potentials are measured in the test buffer with ethyl alcohol and compared with the membrane potentials measured in a reference buffer without ethyl alcohol. The ratio of the membrane potential (MP) in the test buffer divided by the MP in the reference buffer is called the membrane potential ratio (MPR™). MPR™ values are significantly different in the three groups (see U.S. application Ser. No. 14/236,787, the disclosure of which is incorporated herein by reference in its entirety).

It is generally well recognized that the mental disorders are caused by the malfunction of the neurons in the brain. Neurons communicate with each other through electro-biological signals. These signals are generated and modulated by the membrane potential (MP) and the excitability of the neurons. It is essential to understand the biological basis for these differences in blood cells in order to establish the relationship of these results to neurons and to elucidate the pathophysiology of these illnesses. It is the objective of this effort to discover the common biological pathway that gives rise to the observed differences.

The identification of the molecules that modulate the signaling pathways in the neuronal cell is essential in diagnosing and treating mental illness. The membrane potential is the electrical potential difference (voltage) across a cell's membrane. Membrane potential results from the action of K⁺ ion channels present in the membrane which along with the Na, K-ATPase enzyme maintain viable ion concentrations inside the cell.

Unlike most cells, neurons are electrically active and use changes in membrane potential for fast communication with other neurons. Neurons process and transmit information in the form of electrical signals, K⁺ ion channels in the neuronal membrane set the membrane potentials and the excitability. These signals are then processed, amplified and transmitted to the synapse releasing the neurotransmitters. These transmitters again send a signal through their specific g-protein coupled receptors (GPCR) in the membrane of the target neuron. The GPCRs transmit these signals through two primary signal transduction pathways that process and transmit this signal to the K⁺ ion channels in its membrane. These two pathways are the cAMP signaling pathway and the DAG signaling pathway (Nahorski S. R. British Journal of Pharmacology (2006) 147, S38-S45, the disclosure of which is incorporated herein by reference in its entirety).

Calculations of the membrane potentials (MP) using Goldman-Hodgkin-Katz equation showed that lithium would depolarize the membrane potentials (Thiruvengadam, A. Journal of Affective Disorders 65 (2001) 95-99, the disclosure of which is incorporated herein by reference in its entirety). This result led to the hypothesis that lithium's therapeutic efficacy was due to this depolarizing effect. This result was supported by earlier experimental and clinical results (Yonemura, K, and Sato, M, The Japanese Journal of Physiology, 1967; 17: 678-97; Grafe, et al, Brain Research, 1983; 279: 65-76 and El-Mallakh, et al, J. Affective Disorders, 1996; 41: 33-3; the disclosures of which are incorporated herein by reference in their entirety). Thiruvengadam (Focus on Bipolar Disorder Research ISBN 1-59454-059-4 Editor: Malcomb R. Brown, pp. 15-35, 2005 Nova Science Publishers, Inc.; the disclosure of which is incorporated herein by reference in its entirety) further showed that lithium not only depolarizes the MP but also reduced the excitabilities of neurons. Measurement of membrane potentials of cultured lymphoblasts collected from BD patients showed that the MP was hyperpolarized confirming the measurements of El Mallakh et al. In order to use the MP as a diagnostic marker for BD, a ratiometric method was developed and used successfully for diagnosing BD patients (U.S. Pat. No. 7,425,410B2; the disclosure of which is incorporated herein by reference in its entirety) using their red blood cells (RBC) This Method involves the measurement of MP in two buffers and taking the ratio of these two MPs. These experiments involve a test buffer that contains no K+ ions but contains ethyl alcohol (EtOH). The membrane potentials are measured in the test buffer and compared with the membrane potentials measured in a reference buffer without EtOH. This ratio is called the Membrane Potential Ratio (MPR™). It was further discovered that the MPR™ could also be used to diagnose the ADHD patients (U.S. Pat. No. 7,906,300B2; the disclosure of which is incorporated herein by reference in its entirety).

To date, more than 550 patients have been tested using the MPR™. A summary of these test results is shown in FIG. 1. The MPR™ values for BD patients were significantly lower than that for Negatives (including normals, unipolar depressives, and schizophrenics); on the other hand, the MPR™ values for ADHD patients were significantly higher than that for Negatives as shown in FIG. 1.

It is essential to understand the biological basis for these differences in order to establish the scientific mechanisms and the pathways responsible for the differences in the MPR™ among the three groups and to elucidate the pathophysiology of these illnesses.

These signaling pathways and polypeptides can then be used for diagnostic and therapeutic purposes. For example, this invention traces the pathway for BD and ADHD from the G-protein Controlled Receptors (GPCR) to the K⁺ channel in patients' cell. As described in U.S. application Ser. No. 14/236,787, this discovery provided a better understanding of the pathophysiology of these disorders.

CAK Channels and Membrane Potentials in RBC: Although the expression of one of the small conductance family of CAK channels in RBC has been known since 2003 (Hoffman et al, PNAS2003vol. 100 no. 12: 7366-7371), there is no prior art of measuring the MP in RBC leave alone observing the differences among the three groups of patient populations (Negatives, BD and ADHD). Those skilled in the art recognize that the observation that EtOH hyperpolarizes the membrane potentials is a new discovery. Only the experiments using channel blockers, quinine and clotrimazole in RBC established this fact. Patent search as well as literature search using the key words CAK channels and EtOH did not yield any results, CAK channels and MP also did not yield any patents. Adelman et al patent (hSK₂ Channels Adelman et al U.S. Pat. No. 6,797,486) is concerned about hSK₂ DNA sequence and its effect on K⁺ flow throw the channel. Gene sequencing of the hSK4 genes from blood samples drawn from patients did not yield any mutations in the DNA sequence which could explain the MPR™ differences (unpublished results on file).

Ca²⁺/CaM Activation of CAK Channels, EtOH and Membrane Potentials in RBC: CAK channels are activated by Ca²⁺/CaM is well known in the literature. But it is not obvious from the literature that the membrane potentials can be modulated by either EtOH or by a CaM activator such as CaM Kinase II. A patent search using CaM Kinase II and membrane potentials did not yield any results.

PKC, CaM and membrane potentials: It is well known that PKC through the DAG signaling pathway activates the CaM. However there is no literature indicating that DAG signaling pathway modulates the CaK channels and MP. Those skilled in the art recognize that this is an important discovery.

DAG, CAK Channels and MP: It is not at all known in the published literature that the DAG has any effect on membrane potentials leave alone in BD and ADHD. There are no patents connecting DAG, MP, BD and ADHD. Caricasole, et al. (DGK Beta Patent #6,593,121 2003) do not address the MPR™ differences and the DAG Pathway that modulates the MPR™. A genome-wide association study implicated the diacylglycerol kinase eta (DGKH) and several other genes in the etiology of bipolar disorder (Baum et al, Mol Psychiatry. 2008 February; 13(2): 197-207). While this study supports this invention it does not a priori recognize the MPR™ as the connecting link via the DAG signaling pathway.

The buffers that may be used in the diagnostic and agent identifying methods of the present invention include, but are not limited to, the buffers described in U.S. Pat. Nos. 7,425,410 and 7,906,300 which are hereby incorporated by reference in their entirety. These buffers include regular K-containing buffer which is a HEPES buffer to which potassium has also been added (5 mM KCl, 4 mM NaHCO₃, 5 mM HEPES, 134 mM NaCl, 2.3 mM CaCl₂, and 5 mM glucose) and is also referred to as “regular” or “stock” buffer at a pH of 7.4 (range of 7.3 to 7.5). The assay uses a reference buffer or regular buffer and a test buffer. The “reference buffer” or “regular buffer” contains only Na⁺, Ca²⁺, and HEPES without any other reagents. The “test buffer” containing no potassium (K⁺-free buffer) is a HEPES buffer without potassium (4 mM NaHCO₃, 5 mM HEPES, 134 mM NaCl, 2.3 mM CaCl₂, and 5 mM glucose) and with a K⁺ channel altering agent, at a pH of 6.8 (range of 6.6 to 7.0). The test buffer may also contain 30 μM ethacrynic acid dissolved in EtOH as solvent.

K⁺ channel altering agents include, but are not limited to, ethanol, amphetamine, ephedrine, cocaine, caffeine, nicotine, methylphenidate, lithium, δ-9-tetrahydrocannibinol, phencyclidine, lysergic acid diethylamide (LSD) mescaline, or combinations thereof. Preferably, the K⁺ channel altering agent is ethanol.

When the cells are suspended in a K⁺ free buffer the intracellular K leaks out. However the Na⁺K⁺-ATPase pump cannot compensate for this loss by bringing in the K+ from outside the cell since there is no K⁺ outside. This causes the K⁺ channel to shut down. When a K⁺ channel altering agent (such as ethanol) is added, the agent affects the K⁺ channel, for instance, by opening the K⁺ channel, thus further reducing the membrane potential. This opening depends on the patients from whom the cells were drawn. This difference is reflected in the MPR™ obtained as well as in the pathway governing the cell membrane potentials and excitabilities of the excitable cells.

The present methods provide for an increase in the therapeutic efficacy of a CNS stimulant for ADHD. The present invention unexpectedly found that, an increase in the therapeutic efficacy of such CNS stimulant could be achieved in a combination therapy. The combination therapy allows for a reduction in the dose required to achieve a therapeutic effect for the CNS stimulant, and this reduces, ameliorates or prevents the side effects associated with CNS stimulant treatment. In particular, the CNS stimulant is methylphenidate or an amphetamine.

A combination therapy of the present invention includes methylphenidate and an adjunctive agent.

A combination therapy of the present invention includes an amphetamine and an adjunctive agent.

Just as lithium depolarizes the membrane, methylphenidate hyperpolarizes the membrane as shown U.S. application Ser. No. 14/888,720, the disclosure of which is herein incorporated by reference in its entirety. Furthermore, as shown in U.S. Pat. No. 9,523,673, the disclosure of which is herein incorporated by reference in its entirety, the signaling pathway controls the membrane potential ratio. As discussed herein, the cholinergic agonists increase the formation of DAG and anticholinergic agents decrease the formation DAG (Kaplan H I, Sadock B J. Synopsis of Psychiatry. 8th Ed. Baltimore: Williams & Wilkins 1988:103-104; the disclosure of which is herein incorporated by reference in its entirety). Therefore, a synergic combination of methylphenidate with an anticholinergic agent would enhance the effect of methylphenidate thereby reducing the dosage needed for efficacy in ADHD.

Methylphenidate (MPH) Side Effects and Potential for Addiction

MPH is a commonly used drug for the treatment of ADHD. MPH recommended dose is 10-60 mg daily given in 2 or 3 divided doses. Serious side effects may include stomach pain, nausea, vomiting, loss of appetite, vision problems, dizziness, mild headache, sweating, mild skin rash, numbness, tingling, or cold feeling in your hands or feet, nervous feeling, sleep problems (insomnia), and weight loss. MPH can be very addictive, especially when misused or taken via alternate methods, such as by injection or snorting. The Drug Enforcement Administration (DEA) has classified MPH as a Schedule II drug, meaning it has a high potential for abuse.

The present methods provide for an increase in the therapeutic efficacy of lithium. In particular, the present invention unexpectedly found that, an increase in the therapeutic efficacy of lithium could be achieved in a combination therapy. The combination therapy allows for a reduction in the dose required to achieve a therapeutic effect for lithium, and this reduces, ameliorates or prevents the side effects associated with lithium treatment.

A combination therapy of the present invention includes a lithium compound and an adjunctive agent.

The adjunctive agent may include, but is not limited to, a cholinergic agent, an immunomodulatory agent, a mood stabilizer agent, an antidepressant agent, an anticonvulsant agent, an antipsychotic agent, and an anxiolytic agent.

A cholinergic agent may include, but is not limited to, a direct cholinergicagonist that binds selectively or non-selectively to a muscarinic or nicotinic receptor and an indirect cholinergic agonist.

An indirect cholinergic agonist may include, but is not limited to, an acetylcholinesterase inhibitior and aM2receptor antagonist. An acetylcholinesterase inhibitor may include, but is not limited to, donezpezil, galantamine, rivastigminc, tacrine, donepezil/memantine, and pharmaceutically acceptable salts thereof. A M2 receptor antagonist may include, but is not limited to, methoctramine, AF-DX384, and pharmaceutically acceptable salts thereof. an agent that increases the presence of acetylcholine at a muscarinic or nicotinic receptor.

A direct cholinergic agonist that binds selectively or non-selectively to a M1 to M5muscarinic receptor may include, but is not limited to, acetylcholine, methacholine, arecoline, bethanechol, carbachol, pilocarpine, muscarine, cevimeline, nicotine, and pharmaceutically acceptable salts thereof.

An immunomodulatory agent may include, but is not limited to, levamsiole and pharmaceutically acceptable salts thereof.

A mood stabilizer agent, may include, but is not limited to, valproate, divalproex, carbamazepine, lamotrigine, oxacarabazepine, and pharmaceutically acceptable salts thereof.

An anticonvulsant agent, may include, but is not limited to, lamotrigine, perampanel, mephobarbital, primidone, phenobarbital, diazepam, clonazepam, lorazepam, clobazam, felbamate, topiramate, acetazolamide, zonisamide, rufinamide, oxcarbazepine, carbamazepine, eslicarbazepine, valproic acid, divalproex sodium, gabapentin, gabapentin enacarbil, tiagabine, phenytoin, fosphenytoin, mephenytoin, ethotoin, magnesium sulfate, lacosamide, ezogabine, trimethadione, levetiracetam, ethosuximide, methsuximide, and pharmaceutically acceptable salts thereof.

An antidepressant agent may include, but is not limited to, fluoxetine, ariprazole, doxepin, clomipramine, bupropion, amoxapine, nortriptyline, vortioxetine, citalopram, duloxetine, trazodone, venlafaxine, selegiline, perphenazine, amitriptyline, levomilnacipram, desvenlafaxine, lurasidone, lamotrigine, escitalopram, chlordiazepoxide, isocarboxazid, phenelzine, desipramine, trazodone, tranylcypromine, paroxetine, mirtazapine, quetiapine, nefazodone, doxepin, trimipramine, imipramine, vilazodone, protriptyline, sertraline, olanzapine, and pharmaceutically acceptable salts thereof.

An anxiolytic agent may include, but is not limited to, secobarbital, mephobarbital, pentobarbital, phenobarbital, amobarbital, butabarbital, estazolam, alprazolam, flurazepam, diazepam, chlordiazepoxide, clorazepate, clonazepam, oxazepam, diazepam, triazolam, lorazepam, temazepam, midazolam, clobazam, diphenhydramine, zolpidem, chloral hydrate, doxepin, sodium oxybate, doxylamine, doxepin, hydroxyzine, meprobamate, ethchlorvynol, eszopiclone, buspirone, zalephon, ramelteon, suvorexant, tryptophan, tasimelteon, dexmedetomidine, and pharmaceutically acceptable salts thereof.

An antipsychotic agent, may include, but is not limited to, haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof.

In some embodiments, the cholinergic agonist may be, for example, one or more of acetylcholine, nicotine, muscarine, carbachol, galantamine, arecoline, cevimeline, levamisole, clozapine and donepezil.

As used herein, “an effective amount,” “a therapeutically effective amount” or “an effective dosage” is one which reduces symptoms of the BD condition or pathology, and preferably which normalizes physiological responses in an individual with the BD condition or pathology. MPR™ may be used to identify the “effective amount,” the therapeutically effective amount” or the “effective dosage” directly through a blood test. For instance, the effective amount of an amount of lithium and/or the effective amount of an adjunctive agent is an amount which brings the diagnostic probability to the negative range as discussed U.S. application Ser. No. 14/236,787, the disclosure of which is incorporated herein in its entirety. As exemplified in Example 4 below, in a BD patient, the MPR™ returns to negative with treatment using an effective amount. This an example of how an “effective amount” or “effective dosage” can be determined.

Reduction of symptoms or normalization of physiological responses can be determined using methods routine in the art for assessing BD. In one aspect, “an effective amount” or a “therapeutically effective amount” of a lithium compound and/or “an effective amount” or a “therapeutically effective amount” of at least one adjunctive agent of the invention, or a pharmaceutical combination or composition comprising the same of the invention, is an amount which restores a measurable physiological parameter, such as the membrane potential, to substantially the same value (for instance, preferably to within 30% or less, more preferably to within 20% or less, and still more preferably, to within 10% or less) of the value of the parameter in an individual without BD disease condition or pathology. In another aspect, “an effective amount” or a “therapeutically effective amount” of a lithium compound and/or “an effective amount” or a “therapeutically effective amount” of at least one adjunctive agent of the invention, or a pharmaceutical combination or composition comprising the same of the invention, is an amount which restores a measurable physiological parameter, such as the membrane potential, to a value substantially higher than (preferably at least 10% higher than, more preferably at least 20% higher than, and still more preferably at least 30% higher than) the parameter of a BD control individual. The percentage may be determined by a clinician treating the patient. The criteria may be whether the effective amount brings down the diagnostic probability to the negative range. The dosage may be adjusted or vary according to the patient response to lithium and/or an adjunctive agent, or the patient response to the synergistic combination.

In one embodiment, an “effective amount” or “therapeutically effective amount” may be associated with an amount sufficient to provide a therapeutically efficacious plasma level of a drug, as may be determined during clinical treatment. A “therapeutically efficacious plasma level” is the amount of the drug (such as a lithium compound or an adjunctive agent) present in the blood sufficient to produce a therapeutic effect.

For instance, an “effective amount” or “therapeutically effective amount” may be associated with an amount sufficient to provide a plasma lithium level of 2.0 mM or less, preferably a plasma lithium level of 1.2 mM or less, preferably a plasma lithium level of 1 mM or less, a plasma lithium level of from 0.5 mM to 1.2 mM, a plasma lithium level of from 0.8 mM to 1.2 mM, more preferably, a plasma lithium level of from 0.6 mM to 0.75 mM, or more preferably a plasma lithium level of from 0.4 mM to 0.6 mM. More preferably, an “effective amount” or “therapeutically effective amount” of a lithium compound, may be associated with an amount sufficient to provide a plasma lithium level of at least 1 mM, a plasma lithium level of at least 0.8 mM, preferably, a plasma lithium level of at least 0.5 mM, or a plasma lithium level of at least 0.4 mM. This effective amount or therapeutically effective amount may be determined clinically, and the amount of lithium or adjunctive agent sufficient to provide the above plasma lithium levels may be an amount less than that used in current BD drug therapy, since certain drugs described herein may increase the DAG concentration by 10 fold.

In another embodiment, an “effective amount” or “therapeutically effective amount” may be associated with an amount sufficient to provide a plasma lithium level of 2.0 mEq/L or less, preferably a plasma lithium level of 1.2 mEq/L or less, a plasma lithium level of 1 mEq/L or less, a plasma lithium level of from 0.5 mEq/L to 1.2 mEq/L, a plasma lithium level of from 0.8 mEq/L to 1.2 mEq/L, more preferably, a plasma lithium level of from 0.6 mEq/L to 0.75 mEq/L, or more preferably a plasma lithium level of from 0.4 mEq/L to 0.6 mEq/L More preferably, an “effective amount” or “therapeutically effective amount” of a lithium compound, may be associated with an amount sufficient to provide a plasma lithium level of at least 1 mEq/L, a plasma lithium level of at least 0.8 mEq/L, preferably, a plasma lithium level of at least 0.5 mEq/L, or a plasma lithium level of at least 0.4 mEq/L. This amount may be determined clinically, and may depend on the adjunctive drug used with lithium in a drug combination, so that the effective amount may be determined to be associated with a plasma lithium level as low as 0.1 mEq/L (up to 1.2 mEq/L). Preferably, the effective amount of lithium in the drug combination of the present invention, is an amount less than that used in current BD drug therapy.

Likewise, as is apparent to one skilled in the art, an “effective amount” or “therapeutically effective amount” of an adjunctive agent described herein may be associated with an amount sufficient to provide a therapeutically efficacious plasma level of the respective adjunctive agent. This amount may also be determined through clinical treatment. The “effective amount” or “therapeutically effective amount” amount of an adjunctive agent maybe determined based on a plasma lithium level as described above. Preferably, the effective amount of an adjunctive agent in the drug combination of the present invention, is an amount less than that used in its current drug therapy.

The “effective amount,” “therapeutically effective amount” or the “effective dosage” may be an amount of lithium that is sufficient to interact synergistically with at least one adjunctive agent, to improve or enhance the therapeutic effect or therapeutically efficacious plasma level of the adjunctive agent; and/or an amount of at least one adjunctive agent that is sufficient to interact synergistically with lithium to improve or enhance the therapeutic effect or therapeutically efficacious plasma level of lithium.

Non-limiting examples of therapeutically efficacious plasma levels of adjunctive agents useful in the present invention are exemplified below.

Amitriptyline: 120 to 150 ng/mL

Carbamazepine: 5 to 12 μg/mL
Nortriptyline: 50 to 150 ng/mL
Phenobarbital: 10 to 30 μg/mL
Phenytoin: 10 to 20 μg/mL
Valproic acid: 50 to 100 μg/mL

Preferably, the effective amount of lithium compound is a dose amount that is less than a dosage of lithium required to provide a therapeutic effect in current BD therapy when used alone, or is a dose amount that is less than a dosage of lithium required in current BD therapy when used alone to provide a therapeutically efficacious plasma lithium level for BD therapy. For instance, the effective dose may be a dose that brings the diagnostic probability to the negative range. Likewise, the effective amount of at least one adjunctive agent may include a dose that is less than a dosage of the at least one adjunctive agent required to provide a therapeutically efficacious plasma level of the at least one adjunctive agent when administered alone.

As is apparent to one skilled in the art, an “effective amount” or a “therapeutically effective amount” of a lithium compound and/or “an effective amount” or a “therapeutically effective amount” of at least one adjunctive agent of the invention, or a pharmaceutical combination or composition comprising the same of the present invention, will also vary depending upon the age, weight and mammalian species treated, the particular compounds employed, the particular mode of administration and the desired effects and the therapeutic indication. Because these factors and their relationship to determining this amount are well known, the determination of an effective dosage level or therapeutically effective dosage levels ˜such as the amount necessary to achieve the desired result therapeutically efficacious plasma level of lithium or therapeutically efficacious plasma level of an adjunctive agent described herein− will be within the skill of the skilled person. Alternatively, the determination of an effective dosage level or therapeutically effective dosage levels—the amount which restores a measurable physiological parameter such as the membrane potential to substantially the same value to the negative range as exemplified in Example 4 (preferably to within 30% or less, more preferably to within 20% or less, and still more preferably, to within 10% or less) of the value of the parameter in an individual without BD disease condition or pathology, or the amount which restores a measurable physiological parameter, such as the membrane potential, to a value substantially higher than (preferably at least 10% higher than, more preferably at least 20% higher than, and still more preferably at least 30% higher than) the parameter of a BD control individual—will be within the skill of the skilled person.

For instance, an “effective amount” or a “therapeutically effective amount” of a lithium compound or of at least one adjunctive agent of the present invention, or a pharmaceutical combination or composition of the present invention, will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy so as to deliver the agent, pharmaceutical combination, or pharmaceutical composition to the BD patient, but will depend on such factors as weight, diet, concurrent medication and other factors, well known to those skilled in the medical arts.

In some combination therapies of the present invention, the combination or composition comprising lithium and the at least one cholinergic agonist may be present together in a single dosage form, or may be present in separate dosage forms. For different patients, and even for the same patient over time (for example, if the symptoms of bipolar disorder improve or worsen; or for example, depending on the result of a mean membrane potential test from cells obtained from a BD patient during or after therapy), the dosage of lithium and/or the at least one cholinergic agonist may be increased or decreased. The process of adjusting dosages in an upward or downward direction and evaluating the effect of the adjustment on mean membrane potential, and/or BD symptoms, may be continued until an optimum dosage is determined to bring the diagnostic probability to the negative range at which the patient experiences the best balance between therapeutic effectiveness and side-effects.

Dosages of the lithium compound and at least one adjunctive agent (such as a cholinergic agonist) may vary depending on such factors as, for example, the characteristics of the patient, and the frequency of administration.

The at least one adjunctive agent (such as a cholinergic agonist) may be administered such that the patient is provided with a therapeutically-effective plasma concentration thereof. In some embodiments, where the cholinergic agonist is carbachol, the patient may be provided with a plasma concentration of 30 μM or less, 25 μM or less, 20 μM or less, 15μM or less, 10 μM or less, 9 μM or less, 8 μM or less, 7 μM or less, 6 μM or less, 5 μM or less, 4 μM or less, 3 μM or less, or 2 μM or less. Alternatively, the optimum concentration may be determined based on the patient's individual factors or may be determined through patient clinical trials using the diagnostic probability as the criterion as described earlier.

In some embodiments, where the cholinergic agonist is clozapine, the patient is provided with a plasma concentration of 500 ng/ml or less, 400 ng/ml or less, 300 ng/ml or less, 200 ng/mi or less, 150 ng/ml or less, 100 ng/ml or less, 90 ng/mi or less, 80 ng/ml or less, 70 ng/ml or less, 60 ng/ml or less, 50 ng/ml or less, 40 ng/ml or less, 30 ng/ml or less, 20 ng/ml or less, or 10 ng/ml or less. Alternatively, the optimum concentration may be determined based on the patient's individual factors or may be determined through patient clinical trials using the diagnostic probability as the criterion as described earlier.

In some embodiments, where the cholinergic agonist is donepezil, the patient is provided with a plasma concentration of 50 ng/mi or less, 40 ng/ml or less, 30 ng/ml or less, 20 ng/ml or less, 10 ng/ml or less, 9 ng/ml or less, 8 ng/ml or less, 7 ng/ml or less, 6 ng/ml or less, 5 ng/ml or less, 4 ng/ml or less, 3 ng/ml or less, or 2 ng/ml or less. Alternatively, the optimum concentration may be determined based on the patient's individual factors or may be determined through patient clinical trials using the diagnostic probability as the criterion as described earlier.

The biochemical form of lithium is not strictly limited. In some embodiments, the lithium may be in the form of lithium carbonate. However, other salt forms that could serve as a source of lithium include, for example: lithium benzoate, lithium bromide, lithium cacodylate, lithium caffeine sulfonate, lithium chloride, lithium citrate, lithium dithiosalicylate, lithium formate, lithium glycerophosphate, lithium iodate and lithium salicylate. The lithium salts may be given in a substantially pure form or mixed with other compounds, foods, or therapeutic agents as the exigencies of individual cases require.

The lithium and/or the at least one adjunctive agent (such as a cholinergic agonist) of the combination therapy of the present invention may be administered separately or together, with or without a pharmaceutically acceptable carrier or vehicle. They can be provided in dosage forms such as tablets, capsules, powder packets, or liquid solutions for oral administration. Methods for preparing these dosage forms are well known in the art (see. e.g., Remington's Pharmaceutical Sciences, 16th Ed., A. Oslo Ed. Mack, Easton, Pa. (1980), incorporated herein by reference in its entirety). When given orally, therapeutically inert agents may be added to improve palatability, or additional therapeutic agents may be added. Pharmaceutically acceptable carriers include diluents and excipients generally used in pharmaceutical preparations, such as fillers, extenders, binders, moisturizers, disintegrators, surfactants, and lubricants. The lithium and/or the at least one cholinergic agonist of the combination therapy of the present invention may be formulated as a pharmaceutical preparation, for example in the form of tablets, flash melt tablets, pills, powder, liquid, suspension, emulsion, granules, capsules, suppositories or injection (liquid, suspension, etc.), troches, intranasal spray percutaneous patch and the like.

In case of shaping to tablet formulation, a wide variety of carriers that are known in this field can be used. Examples include lactose, saccharose, sodium chloride, glucose, urea, starch, xylitol, mannitol, erythritol, sorbitol, calcium carbonate, kaolin, crystalline cellulose, silic acid and other excipients; water, ethanol, propanol, simple syrup, glucose solution, starch solution, gelatin solution, carboxymethyl cellulose, shellac, methyl cellulose, potassium phosphate, polyvinyl pyrrolidone and other binders; dried starch, sodium alginate, agar powder, laminaran powder, sodium hydrogencarbonate, calcium carbonate, polyoxyethylenesorbitan fatty acid esters, sodium lauryl sulfate, stearic acid monoglyceride, starch, lactose and other disintegrators; white sugar, stearin, cacao butter, hydrogenated oil and other disintegration inhibitors; quaternary ammonium salt, sodium lauryl sulfate and other absorption accelerator; glycerine, starch and other moisture retainers; starch, lactose, kaolin, bentonite, colloidal silic acid and other adsorbents; and refined talc, stearate, boric acid powder, polyethylene glycol and other lubricants and the like. Tablets can also be formulated if necessary as tablets with ordinary coatings, such as sugar-coated tablets, gelatin-coated tablets, enteric coated tablets and film coated tablets, as well as double tablets and multilayered tablets.

In case of shaping to pills, a wide variety of carriers that are known in this field can be used. Examples include glucose, lactose, starch, cacao butter, hardened vegetable oil, kaolin, talc and other excipients; gun arabic powder, traganth powder, gelatin, ethanol and other binders; and laminaran, agar and other disintegrators and the like.

In case of shaping to a suppository formulation, a wide variety of carriers that are known in the field can be used. Examples include polyethylene glycol, cacao butter, higher alcohol, esters of higher alcohol, gelatin semi-synthetic glyceride and the like.

In addition, colorants, preservatives, perfumes, flavorings, sweeteners and the like as well as other drugs may be contained in the pharmaceutical composition.

Individual preparations of a cholinergic agonist and lithium may also be provided in the form of a kit, comprising a carrier (e.g. a box or bag) compartmentalized to receive one or more components (bottles, vials, packets etc.) in close confinement. It is expected that such a kit would be carried by patients with bipolar disorder and that it would contain written instructions concerning the way in which the enclosed drugs should be taken, potential side effects, etc. The kit should be portable, and be generally convenient for use by patients.

For parenteral administration, preparations containing lithium and/or at least one cholinergic agonist may be provided to patients in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles include fluid and nutrient replenishers, electrolyte replenishers, such as those based upon Ringer's dextrose and the like.

The methods for administration of the pharmaceutical composition of the present invention are not specifically restricted. The composition is administered depending on each type of preparation form, and the age, gender and other condition of the patient (degree and conditions of the disease, etc.). For example, tablets, pills, liquids, suspensions, emulsions, granules and capsules are administered orally. In case of injection preparation, it is administered intravenously either singly or mixed with a common auxiliary liquid such as solutions of glucose or amino acid. Further, if necessary, the injection preparation is singly administered intradermally, subcutaneously or intraperitoneally. In case of a suppository, it is administered intrarectally.

The patient may be administered the combination therapy several times per day, once per day, once every other day, or once per week or less. The lithium compound and at least one adjunctive agent contemplated herein may be administered, simultaneously with or sequentially (such as prior to or after), in combined or separate formulation(s), in a coordinate treatment protocol. In certain embodiments, a lithium compound is administered coordinately with at least one adjunctive agent contemplated herein, using separate formulations or a combinatorial formulation as described herein (i.e., comprising both a lithium compound, and at least one adjunctive agent). This coordinate administration may be done simultaneously or sequentially in either order, and there may be a time period while only one or both (or all) active therapeutic agents individually and/or collectively exert their biological activities.

The combination therapies of the present invention may include, in addition to lithium and at least one adjunctive agent such as, one or more of 1) mood stabilizers such as Cibalith, Eskalith, Lithane, Litho-tabs, and Lithobid; 2) anti-psychotics such as Abilify, Geodon, Haldol, Risperdol, Saphris, Seroquel, Zyprexa, and Symbyax; 3) anti-anxiety Drugs such as Ativan, Klonopin, Valium, and Xanax; and/or 4) anti-convulsants such as Depakote, Lamictal, and Tegretol.

In the present invention, one method for determining the optimum dose of a combination therapy for the treatment of RD, or for monitoring the efficacy of a combination therapy for the treatment of BD, is to determine the membrane potential ratio (MPR™) of cells obtained from the BD patient. The MPR™ test has been described in U.S. Pat. Nos. 7,425,410 and 7,906,300, as well as U.S. Provisional Application Nos. 61/543,061 and 61/653,579, which are hereby incorporated by reference in their entirety. Briefly, the MPR™ test involves measuring the membrane potential of the human cells in a test buffer and in a reference buffer, and calculating the ratio of these membrane potentials. U.S. Pat. Nos. 7,425,410 and 7,906,300 describe the use of this method to diagnose BD; however, it can also be used to determine the optimum dose of a combination therapy for the treatment of BD, or to monitor the efficacy of a combination therapy for the treatment of BD, by measuring and/or adjusting the MPR™ values. For example, in some embodiments, if the BD patients respond to the combination therapy then the MPR™ values return to the negative range. Otherwise the treatment protocol is adjusted appropriately till the MPR™ values reach the negative range.

The membrane potentials of whole blood cells can be measured using two different buffers in a plate reader. The mean MPR™ value is the ratio between the membrane potential of a patient's cells in the test buffer as the numerator and that in the reference buffer as the denominator (for example, determined by statistical analysis of multiple measurements, using the ANOVA and the multiple statistical regression analysis). See Thiruvengadam et al., J. Affect Disord 100(1-3):75-82 (2007), which is hereby incorporated by reference in its entirety.

In some aspects, the present invention relates to determining the optimum dose of a combination therapy for the treatment of BD, by analyzing the membrane potential of cells isolated from a BD patient treated with the combination therapy, and calculating a membrane potential ratio therefrom.

### First Embodiment

In one embodiment, a method of determining an optimal combination drug treatment therapy for a patient with attention deficit hyperactivity disorder (ADHD), is provided that comprises:

obtaining a ratio of a mean membrane potential that is a mean membrane potential of a first population of cells from the ADHD patient incubated in vitro in the presence of an agent that alters diacylglycerol signaling and in the absence of K⁺, to a mean membrane potential of a second population of cells from the ADHS patient incubated in vitro in the absence of the test agent that alters diacylglycerol signaling and in the presence of K⁺ or absence of K⁺ (preferably, the test buffer is in the absence of K⁺ (i.e., both reference buffer and test buffer do not have K⁺); comparing the ratio of the mean membrane potential to (a) and/or (b):
(a) a control ratio of a mean membrane potential of first population of control human cells known to not have ADHD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+,
(b) an ADHD control ratio of a mean membrane potential of first population of bipolar control human cells known to have ADHD incubated in vitro in the presence of the agent that alters diacylglycerol signaling and in the absence of K⁺, to a mean membrane potential of a second population of the bipolar control human cells incubated in vitro in the absence of the agent that alters diacylglycerol signaling and in the presence of K⁺ or absence of K⁺;

identifying the optimal combination drug treatment therapy when the ratio of the mean membrane potential obtained is not significantly different from the control ratio of (a), is decreased towards the control ratio (a) in comparison to or relative to the ADHD control ratio of (b), and/or is significantly lower or decreased in comparison to or relative to the ADHD control ratio of (b).

The method may further include obtaining an initial ratio of a mean membrane potential from an initial population of cells from the human patient before the obtaining step.

The human cells that may be used in the present method include, but is not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise a CNS stimulant and at least one adjunctive agent. The CNS stimulant may be an amphetamine, and preferably, is methylphenidate.

The amphetamine may include, but is not limited to, dextroamphetamine, levoamphetamine, lisdexamfetamine, methamphetamine, Adderall® (amphetamine and dextroamphetamine mixed salts), Adderall XR® (amphetamine and dextroamphetamine mixed salts), Dexedrine® (dextroamphetamine sulfate), ProCentra® (dextroamphetamine sulfate), Dextrostat® (dextroamphetamine sulfate), Ritalin® (methylphenidate hydrochloride), Concerta® (methylphenidate extended release), Vyvanse® (lisdexamfetamine dimesylate), Focalin® (dexmethylphenidate hydrochloride), and Strattera® (atomoxetine hydrochloride).

Preferably, the effective amount of the CNS stimulant is a dose amount that is less than a dosage of the CNS stimulant required to provide a therapeutic effect for ADHD therapy when used alone, or is a dose amount that is less than a dosage of the CNS stimulant required to provide a therapeutically efficacious plasma level of the CNS stimulant for ADHD therapy when used alone. For instance, the effective dose may be a dose that brings the diagnostic probability to the negative range. Preferably, the CNS stimulant is methylphendiate.

In one embodiment, the effective amount of methylphenidate is the dosage amount that improves or enhances the therapeutic effect or therapeutically efficacious plasma level of an adjunctive agent.

The at least one adjunctive agent used in the method may include, but is not limited to, an anticholinergic agent. The anticholinergic agent may include, but is not limited to, trihexyphenidyl, benztopine mesylate, ipratropium, tiotropium, orphenadrine, atropine, flavoxate, oxybutynin, scopolamine, methscopolamine, hyoseyamine, tolterodine, festoterodine, solifenacin, darifenacin, propantheline, glycopyrrolate, dicyclomine, and pharmaceutically acceptable salts thereof.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

In a preferred embodiment, the effective amount of an adjunctive agent in the drug combination of the present invention, is an amount less than that used in its current drug therapy.

In another preferred embodiment, the effective amount of an anticholinergic agent is the dosage amount that is sufficient to improve or enhance the therapeutic effect or therapeutically efficacious plasma level of methylphenidate.

The agent that alters diacylglycerol signaling may include, but is not limited to, a calcium-calmodulin (Ca²⁺/CaM) kinase inhibitor, a diacylglycerol kinase inhibitor, a protein kinase C inhibitor, and an agent that affects calcium-activated potassium (CaK) channels.

Preferably, the agent is a calcium-calmodulin (Ca²⁺/CaM) kinase inhibitor, such as autocamtide-2-related inhibitory peptide (AIP).

Preferably, the agent is a diacyglycerol kinase inhibitor, such as 6-[2-[4-[(4-fluorophenyl)phenylmethylene]-1-piperidinyl]ethyl]-7-methyl-5H-thiazolo[3,2-a]pyrimidin-5-one (ALX).

The mean membrane potential test may further include incubating the cells in vitro in buffer comprising a potential-sensitive dye, resuspending the cells in potential-sensitive dye free-buffer, and measuring the cell fluorescence.

The agent that alters K⁺ channel activity may include, but is not limited to, ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

### Second Embodiment

In a second embodiment, the present invention provides a method of optimizing a combination drug treatment therapy for a patient with attention deficit hyperactivity disorder (ADHD), comprising the steps of:

obtaining at least one sample from an ADHD patient in a drug therapy treatment for ADHD;

performing on each sample, a mean membrane potential test comprising:
obtaining a ratio of a mean membrane potential that is a mean membrane potential of a first population of cells from the sample incubated in vitro in the presence of an agent that alters diacylglycerol signaling and in the absence of K⁺, to a mean membrane potential of a second population of the sample incubated in vitro in the absence of the test agent that alters diacylglycerol signaling and in the presence of K⁺ or absence of K⁺;
comparing the ratio of the mean membrane potential to (a) and/or (b):
   (a) a control ratio of a mean membrane potential of a first population of control human cells known to not have ADHD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+.
   (b) an ADHD control ratio of a mean membrane potential of a first population of bipolar control human cells known to have ADHD incubated in vitro in the presence of the agent that alters diacylglycerol signaling and in the absence of K+, to a mean membrane potential of a second population of the bipolar control human cells incubated in vitro in the absence of the agent that alters diacylglycerol signaling and in the presence of K+ or absence of K+;

determining an optimal drug therapy treatment for the ADHD patient when the ratio of the mean membrane potential obtained is not significantly different from the control ratio of (a), is decreased towards the control ratio (a) in comparison to or relative to the ADHD control ratio of (b), and/or is significantly lower or decreased in comparison to or relative to the ADHD control ratio of (b).

The method optionally includes modifying at least one drug in the drug therapy treatment for ADHD when the least one drug treatment therapy for ADHD is determined to not be the optimal drug therapy treatment. Such as when the ratio of the mean membrane potential obtained is higher in comparison to or relative to the control ratio of (a), is increased towards the ADHD control ratio of (b) in comparison to or relative to the control ratio of (a), and/or is not significantly different from the ADHD control ratio in (b).

The method may further include obtaining an initial ratio of a mean membrane potential from an initial population of cells from the human patient before the obtaining step.

The human cells that may be used in the present method include, but is not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise methylphenidate and at least one adjunctive agent. The adjunctive agent is preferably, an anticholinergic agent.

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

In one embodiment, the effective amount of methylphenidate is the dosage amount that improves or enhances the therapeutic effect or therapeutically efficacious plasma level of an adjunctive agent.

The at least one adjunctive agent used in the method may include, but is not limited to, an anticholinergic agent as described herein.

The at least one adjunctive agent used in the method may include, but is not limited to, an anticholinergic agent. The anticholinergic agent may include, but is not limited to, trihexyphenidyl, benztopine mesylate, ipratropium, tiotropium, orphenadrine, atropine, flavoxate, oxybutynin, scopolamine, methscopolamine, hyoscyamine, tolterodine, festoterodine, solifenacin, darifenacin, propantheline, glycopyrrolate, dicyclomine, and pharmaceutically acceptable salts thereof.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

In a preferred embodiment, the effective amount of an adjunctive agent in the drug combination of the present invention, is an amount less than that used in its current drug therapy.

In a preferred embodiment, the effective amount of an anticholinergic agent is the dosage amount that is sufficient to improve or enhance the therapeutic effect or therapeutically efficacious plasma level of methylphenidate.

In a preferred embodiment, the effective amount of an adjunctive agent in the drug combination of the present invention, is an amount less than that used in its current drug therapy.

The agent that alters diacylglycerol signaling may include, but is not limited to, a calcium-calmodulin (Ca²⁺/CaM) kinase inhibitor, a diacylglycerol kinase inhibitor, a protein kinase C inhibitor, and an agent that affects calcium-activated potassium (CaK) channels.

Preferably, the agent is a calcium-calmodulin (Ca²⁺/CaM) kinase inhibitor, such as autocamtide-2-related inhibitory peptide (AIP).

Preferably, the agent is a diacylglycerol kinase inhibitor, such as 6-[2-[4-[(4-fluorophenyl)phenylmethylene]-1-piperidinyl]ethyl]-7-methyl-5H-thiazolo[3,2-a]pyrimidin-5-one (ALX).

The mean membrane potential test may further include incubating the cells in vitro in buffer comprising a potential-sensitive dye, resuspending the cells in potential-sensitive dye free-buffer, and measuring the cell fluorescence.

The agent that alters K⁺ channel activity may include, but is not limited to, ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

### Third Embodiment

In a third embodiment, the present invention provides a method for determining an optimum dosage of a drug in a combination drug treatment therapy for the treatment of attention deficit hyperactivity disorder (ADHD), said method comprising:

obtaining at least one sample from a BD patient treated with a dosage of a drug in a combination therapy;

performing on each sample, a mean membrane potential test comprising:

obtaining a ratio of a mean membrane potential that is a mean membrane potential of a first population of cells from the ADHD patient incubated in vino in the presence of an agent that alters diacylglycerol signaling and in the absence of K⁺, to a mean membrane potential of a second population of cells from the ADHD patient incubated in vitro in the absence of the test agent that alters diacylglycerol signaling and in the presence of K or absence of K⁺;

comparing the ratio of the mean membrane potential to (a) and/or (b):
(a) a control ratio of a mean membrane potential of a first population of cells from a control human known to not have said ADHG incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+,
(b) an ADHD control ratio of a mean membrane potential of a first population of cells from a bipolar control human known to have said ADHD incubated in vitro in the presence of the agent that alters diacylglycerol signaling and in the absence of K+, to a mean membrane potential of a second population of cells from the bipolar control human incubated in vitro in the absence of the agent that alters diacylglycerol signaling and in the presence of K+ or absence of K+;

determining the dosage of the drug in the combination drug treatment therapy is an optimal dosage for treating ADHD in the combination therapy when the ratio of the mean membrane potential obtained is not significantly different from the control ratio of (a), is increased towards the control ratio (a) in comparison to or relative to the ADHD control ratio of (b), and/or is significantly higher in comparison to or relative to the ADHD control ratio of (b).

The method may further optionally include determining the dosage of the drug in the combination drug treatment therapy is not the optimal dosage for treating ADHD in the combination therapy when the ratio of the mean membrane potential obtained is higher in comparison to or relative to the control ratio of (a), is increased towards the ADHD control ratio of (b) in comparison to or relative to the control ratio of (a), and/or is not significantly different from the ADHD control ratio of (b).

The method may further optionally include modifying the dosage of the drug in the combination drug treatment therapy when the dosage of the drug in the combination therapy is determined to be not the optimal dosage for treating ADHD based on the mean membrane potential test.

The method may further include obtaining an initial ratio of a mean membrane potential from an initial population of cells from the human patient before the obtaining step.

The human cells that may be used in the present method include, but is not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise methylphenidate and at least one adjunctive agent.

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

In a preferred embodiment, the effective amount of methylphenidate is the dosage amount that improves or enhances the therapeutic effect or therapeutically efficacious plasma level of an adjunctive agent.

The at least one adjunctive agent used in the method may include, but is not limited to, an anticholinergic agent as described herein.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of methylphenidate.

The at least one adjunctive agent used in the method may include, but is not limited to, an anticholinergic agent as described herein.

The at least one adjunctive agent used in the method may include, but is not limited to, an anticholinergic agent. The anticholinergic agent may include, but is not limited to, trihexyphenidyl, benztopine mesylate, ipratropium, tiotropium, orphenadrine, atropine, flavoxate, oxybutynin, scopolamine, methscopolamine, hyoscyamine, tolterodine, festoterodine, solifenacin, darifenacin, propantheline, glycopyrrolate, dicyclomine, and pharmaceutically acceptable salts thereof.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

In a preferred embodiment, the effective amount of an adjunctive agent in the drug combination of the present invention, is an amount less than that used in its current drug therapy.

In a preferred embodiment, the effective amount of an anticholinergic agent is the dosage amount that is sufficient to improve or enhance the therapeutic effect or therapeutically efficacious plasma level of methylphenidate.

The agent that alters diacylglycerol signaling may include, but is not limited to, a calcium-calmodulin (Ca²⁺/CaM) kinase inhibitor, a diacylglycerol kinase inhibitor, a protein kinase C inhibitor, and an agent that affects calcium-activated potassium (CaK) channels.

Preferably, the agent is a calcium-calmodulin (Ca²⁺/CaM) kinase inhibitor, such as autocamtide-2-related inhibitory peptide (AIP).

Preferably, the agent is a diacylglycerol kinase inhibitor, such as 6-[2-[4-[(4-fluorophenyl)phenylmethylene]-1-piperidinyl]ethyl]-7-methyl-5H-thiazolo[3,2-a]pyrimidin-5-one (ALX).

The mean membrane potential test may further include incubating the cells in vitro in buffer comprising a potential-sensitive dye, resuspending the cells in potential-sensitive dye free-buffer, and measuring the cell fluorescence.

The agent that alters K⁺ channel activity may include, but is not limited to, ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

### Fourth Embodiment

In a fourth embodiment, the present invention provides a method for monitoring the efficacy of a combination drug treatment therapy for the treatment of attention deficit hyperactivity disorder (ADHD), said method comprising:

obtaining at least one sample from an ADHD patient treated with a combination drug treatment therapy for treating ADHD;

performing on each sample, a mean membrane potential test comprising:

obtaining a ratio of a mean membrane potential that is a mean membrane potential of a first population of cells from the ADHD patient incubated in vino in the presence of an agent that alters diacylglycerol signaling and in the absence of K⁺, to a mean membrane potential of a second population of cells from the ADHD patient incubated in vitro in the absence of the test agent that alters diacylglycerol signaling and in the presence of K or absence of K⁺;

comparing the ratio of the mean membrane potential to (a) and/or (b):
(a) a control ratio of a mean membrane potential of a first population of cells from a control human known to not have said ADHD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+,
(b) an ADHD control ratio of a mean membrane potential of a first population of cells from an AHDH control human known to have said AHDH incubated in vitro in the presence of the agent that alters diacylglycerol signaling and in the absence of K+, to a mean membrane potential of a second population of cells from the ADHD control human incubated in vitro in the absence of the agent that alters diacyglycerol signaling and in the presence of K+ or absence of K+;

determining the combination drug treatment therapy is efficacious based on the mean membrane potential test when the ratio of the mean membrane potential obtained is not significantly different from the control ratio of (a), is decreased towards the control ratio in comparison to or relative to the ADHD control ratio of (b), and/or is significantly lower in comparison to or relative to the AHDH control ratio of (b)

The method may optionally further include determining the combination drug treatment therapy is not efficacious based on the mean membrane potential test when the ratio of the mean membrane potential obtained is lower in comparison to or relative to the control ratio of (a), is increased towards the ADHD control ratio of (b) in comparison to or relative to the control ratio of (a), and/or is not significantly different from the ADHD control ratio of (b).

The method may optionally further include adjusting a dosage of one or more agents in the combination drug treatment therapy when the combination therapy is determined to be not efficacious based on the mean membrane potential test.

The method may further include obtaining an initial ratio of a mean membrane potential from an initial population of cells from the human patient before the obtaining step.

The human cells that may be used in the present method include, but is not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise methylphenidate and at least one adjunctive agent.

Preferably, the effective amount of methylphenidate compound is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

In a preferred embodiment, the effective amount of methylphenidate is the dosage amount that improves or enhances the therapeutic effect or therapeutically efficacious plasma level of an adjunctive agent.

The at least one adjunctive agent used in the method may include, but is not limited to, an anticholinergic agent as described herein.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of methylphenidate.

The at least one adjunctive agent used in the method may include, but is not limited to, an anticholinergic agent as described herein.

The at least one adjunctive agent used in the method may include, but is not limited to, an anticholinergic agent. The anticholinergic agent may include, but is not limited to, trihexyphenidyl, benztopine mesylate, ipratropium, tiotropium, orphenadrine, atropine, flavoxate, oxybutynin, scopolamine, methscopolamine, hyoscyamine, tolterodine, festoterodine, solifenacin, darifenacin, propantheline, glycopyrrolate, dicyclomine, and pharmaceutically acceptable salts thereof.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

In a preferred embodiment, the effective amount of an adjunctive agent in the drug combination of the present invention, is an amount less than that used in its current drug therapy.

In a preferred embodiment, the effective amount of an anticholinergic agent is the dosage amount that is sufficient to improve or enhance the therapeutic effect or therapeutically efficacious plasma level of methylphenidate.

The agent that alters diacylglycerol signaling may include, but is not limited to, a calcium-calmodulin (Ca²⁺/CaM) kinase inhibitor, a diacylglycerol kinase inhibitor, a protein kinase C inhibitor, and an agent that affects calcium-activated potassium (CaK) channels.

Preferably, the agent is a calcium-calmodulin (Ca²⁺/CaM) kinase inhibitor, such as autocamtide-2-related inhibitory peptide (A P).

Preferably, the agent is a diacylglycerol kinase inhibitor, such as 6-[2-[4-[(4-fluorophenyl)phenylmethylene]-1-piperidinyl]ethyl]-7-methyl-5H-thiazolo[3,2-a]pyrimidin-5-one (ALX).

The mean membrane potential test may further include incubating the cells in vitro in buffer comprising a potential-sensitive dye, resuspending the cells in potential-sensitive dye free-buffer, and measuring the cell fluorescence.

The agent that alters K⁺ channel activity may include, but is not limited to, ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

### Fifth Embodiment

In a fifth embodiment, the present invention provides a method of treating attention deficit hyperactivity disorder (ADHD), comprising administering an effective amount of a CNS stimulant and at least one adjunctive agent to a human patient with ADHD. The CNS stimulant may include, but is not limited to, an amphetamine and methylphenidate. Preferably, the CNS stimulant is methylphenidate.

The at least one adjunctive agent and CNS stimulant may form a synergistic combination or composition to treat ADHD. Preferably, the at least one adjunctive agent is an anticholinergic agent, and the CNS stimulant is methylphenidate.

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

In a preferred embodiment, the effective amount of methylphenidate is the dosage amount that improves or enhances the therapeutic effect or therapeutically efficacious plasma level of an adjunctive agent.

The at least one adjunctive agent used in the method may include, but is not limited to, an anticholinergic agent as described herein.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of methylphenidate.

The at least one adjunctive agent used in the method may include, but is not limited to, an anticholinergic agent as described herein.

The at least one adjunctive agent used in the method may include, but is not limited to, an anticholinergic agent. The anticholinergic agent may include, but is not limited to, trihexyphenidyl, benztopine mesylate, ipratropium, tiotropium, orphenadrine, atropine, flavoxate, oxybutynin, scopolamine, methscopolamine, hyoscyamine, tolterodine, festoterodine, solifenacin, darifenacin, propantheline, glycopyrrolate, dicyclomine, and pharmaceutically acceptable salts thereof.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

In a preferred embodiment, the effective amount of an adjunctive agent in the drug combination of the present invention, is an amount less than that used in its current drug therapy.

In a preferred embodiment, the effective amount of an anticholinergic agent is the dosage amount that is sufficient to improve or enhance the therapeutic effect or therapeutically efficacious plasma level of methylphenidate.

### Sixth Embodiment

In a sixth embodiment, the present invention provides a method of increasing the therapeutic efficacy of a CNS stimulant for the treatment of attention deficit hyperactivity disorder (ADHD), comprising administering an effective amount of a CNS stimulant with at least one adjunctive agent, to a human patient with ADHD.

The at least one adjunctive agent and the CNS stimulant may form a synergistic combination or composition to treat ADHD.

The CNS stimulant may include, but is not limited to, an amphetamine and methylphenidate. Preferably, the CNS stimulant is methylphenidate.

The adjunctive agent may include, but is not limited to, an anticholinergic agent.

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

In a preferred embodiment, the effective amount of methylphenidate is the dosage amount that improves or enhances the therapeutic effect or therapeutically efficacious plasma level of an adjunctive agent.

The at least one adjunctive agent used in the method may include, but is not limited to, an anticholinergic agent as described herein.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of methylphenidate.

The at least one adjunctive agent used in the method may include, but is not limited to, an anticholinergic agent as described herein.

The at least one adjunctive agent used in the method may include, but is not limited to, an anticholinergic agent. The anticholinergic agent may include, but is not limited to, trihexyphenidyl, benztopine mesylate, ipratropium, tiotropium, orphenadrine, atropine, flavoxate, oxybutynin, scopolamine, methscopolamine, hyoscyamine, tolterodine, festoterodine, solifenacin, darifenacin, propantheline, glycopyrrolate, dicyclomine, and pharmaceutically acceptable salts thereof.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

In a preferred embodiment, the effective amount of an adjunctive agent in the drug combination of the present invention, is an amount less than that used in its current drug therapy.

In a preferred embodiment, the effective amount of an anticholinergic agent is the dosage amount that is sufficient to improve or enhance the therapeutic effect or therapeutically efficacious plasma level of methylphenidate.

### Seventh Embodiment

The invention further provides a method of determining an optimal combination drug treatment therapy for a patient with ADHD, that comprises:

obtaining a ratio of a mean membrane potential that is a mean membrane potential of a first population of cells from the ADHD patient incubated in vitro in the presence of an agent that alters human calcium-activated potassium channels (hSK₄) activity and in the absence of K⁺, to a mean membrane potential of a second population of the human patient cells incubated in vitro in the absence of the test agent that alters human calcium-activated potassium channels (hSK₄) activity and the presence of K⁺ or absence of K⁺;

comparing the test ratio to (a) and/or (b):
(a) a control ratio of a mean membrane potential of control human cells known to not have said ADHD incubated in vitro in the presence of the agent that alters human calcium-activated potassium channels hSK₄ and in the absence of K⁺, to a mean membrane potential of the control human cells incubated in vitro in the absence of the agent that alters human calcium-activated potassium channels hSK₄ and in the presence of K+ or absence of K⁺,
(b) an ADHD control ratio of a mean membrane potential of ADHD control human cells known to have said ADSHD incubated in vitro in the presence of the agent that alters human calcium-activated potassium channels hSK₄ and in the absence of K+, to a mean membrane potential of the ADHD control human cells incubated in vitro in the absence of the agent that alters human calcium-activated potassium channels hSK₄ and in the presence of K⁺ or absence of K⁺;

identifying the optimal combination drug treatment therapy when the ratio of the mean membrane potential is not significantly different from the control ratio of (a), is decreased towards the control ratio (a) in comparison to or relative to the ADHD control ratio of (b), and/or is significantly lower in comparison to or relative to the ADHD ratio of (b).

The method may further include obtaining an initial ratio of a mean membrane potential from an initial population of cells from the human patient before the obtaining step.

The agent that may be used include, but is not limited to, a calcium-calmodulin (Ca²⁺/CaM) kinase inhibitor, a diacylglycerol kinase inhibitor, and a PKC inhibitor. Preferably, the agent is a calcium-calmodulin (Ca²⁺/CaM) kinase inhibitor, such as autocamtide-2-related inhibitory peptide (ALP). In another preferred embodiment, the agent is a diacylglycerol kinase inhibitor such as 6-[2-[4-[(4-fluorophenyl)phenylmethylene]-1-piperidinyl]ethyl]-7-methyl-5H-thiazolo[3,2-a]pyrimidin-5-one (ALX).

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

The human cells that may be used in the present method include, but are not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise methylphenidate and at least one adjunctive agent.

The at least one adjunctive agent used in the method may include, but is not limited to, an anticholinergic agent as described herein.

In a preferred embodiment, the effective amount of methylphenidate is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of methylphenidate.

The at least one adjunctive agent used in the method may include, but is not limited to, an anticholinergic agent. The anticholinergic agent may include, but is not limited to, trihexyphenidyl, benztopine mesylate, ipratropium, tiotropium, orphenadrine, atropine, flavoxate, oxybutynin, scopolamine, methscopolamine, hyoscyamine, tolterodine, festoterodine, solifenacin, darifenacin, propantheline, glycopyrrolate, dicyclomine, and pharmaceutically acceptable salts thereof.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

In a preferred embodiment, the effective amount of an adjunctive agent in the drug combination of the present invention, is an amount less than that used in its current drug therapy.

In a preferred embodiment, the effective amount of an anticholinergic agent is the dosage amount that is sufficient to improve or enhance the therapeutic effect or therapeutically efficacious plasma level of methylphenidate.

The mean membrane potential test may further include incubating the cells in vitro in buffer comprising a potential-sensitive dye, resuspending the cells in potential-sensitive dye free-buffer, and measuring the cell fluorescence.

### Eighth Embodiment

The present invention provides a method of optimizing a combination drug treatment therapy for a patient with attention deficit hyperactivity disorder (ADHD), comprising the steps of:

obtaining a ratio of a mean membrane potential that is a mean membrane potential of a first population of cells from the ADHD patient incubated in vitro in the presence of an agent that alters human calcium-activated potassium channels (hSK₄) activity and in the absence of K⁺, to a mean membrane potential of a second population of the human patient cells incubated in vitro in the absence of the test agent that alters human calcium-activated potassium channels (hSK₄) activity and the presence of K⁺ or absence of K⁺;

comparing the test ratio to (a) and/or (b):
(a) a control ratio of a mean membrane potential of control human cells known to not have said ADHD incubated in vitro in the presence of the agent that alters human calcium-activated potassium channels hSK₄ and in the absence of K⁺, to a mean membrane potential of the control human cells incubated in vitro in the absence of the agent that alters human calcium-activated potassium channels hSK₄ and in the presence of K⁺ or absence of K⁺,
(b) an ADHD control ratio of a mean membrane potential of bipolar control human cells known to have said ADHD incubated in vitro in the presence of the agent that alters human calcium-activated potassium channels hSK₄ and in the absence of K⁺, to a mean membrane potential of the ADHD control human cells incubated in vitro in the absence of the agent that alters human calcium-activated potassium channels hSK₄ and in the presence of K⁺ or absence of K⁺;

determining an optimal drug therapy treatment for the ADHD patient when the ratio of the mean membrane potential obtained is not significantly different from the control ratio in (a), is decreased towards the control ratio in comparison to the ADHD control ratio of (b), and/or is significantly lower than the ADHD control ratio in (b).

The method may further include obtaining an initial ratio of a mean membrane potential from an initial population of cells from the human patient before the obtaining step.

The method may further include optionally modifying at least one drug in the drug therapy treatment for ADHD when the least one drug treatment therapy for ADHD is determined to not be the optimal drug therapy treatment. Such as when the ratio of the mean membrane potential obtained is significantly higher than the control ratio of (a), is increased towards the ASHD control ratio of (b) in comparison to the control ratio of (a), and/or is not significantly different from the ADHD control ratio of (b).

The agent that may be used include, but is not limited to, a calcium-calmodulin (Ca²⁺/CaM) kinase inhibitor, a diacylglycerol kinase inhibitor, and a PKC inhibitor. Preferably, the agent is a calcium-calmodulin (Ca²⁺/CaM) kinase inhibitor, such as autocamtide-2-related inhibitory peptide (AIP). In another preferred embodiment, the agent is a diacylglycerol kinase inhibitor such as 6-[2-[4-((4-fluorophenyl)phenylmethylene]-1-piperidinyl]ethyl-7-methyl-5H-thiazolo[3,2-a]pyrimidin-5-one (ALX).

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

In one embodiment, the effective amount of methylphenidate is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of methylphenidate.

The human cells that may be used in the present method include, but are not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise methylphenidate and at least one adjunctive agent.

The at least one adjunctive agent used in the method may include, but is not limited to, an anticholinergic agent as described herein.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

Preferably, the effective amount of methylphenidate is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of methylphenidate.

The mean membrane potential test may further include incubating the cells in vitro in buffer comprising a potential-sensitive dye, resuspending the cells in potential-sensitive dye free-buffer, and measuring the cell fluorescence.

### Ninth Embodiment

The invention further provides a method of determining an optimum dosage of at least one drug in a combination drug treatment therapy for a patient with ADHD, that comprises:

obtaining a ratio of a mean membrane potential that is a mean membrane potential of a first population of cells from the ADHD patient incubated in vitro in the presence of an agent that alters human calcium-activated potassium channels (hSK₄) activity and in the absence of K⁺, to a mean membrane potential of a second population of the human patient cells incubated in vitro in the absence of the test agent that alters human calcium-activated potassium channels (hSK₄) activity and the presence of K⁺ or absence of K⁺;

comparing the test ratio to (a) and/or (b):
(a) a control ratio of a mean membrane potential of control human cells known to not have said ADHD incubated in vitro in the presence of the agent that alters human calcium-activated potassium channels hSK₄ and in the absence of K⁺, to a mean membrane potential of the control human cells incubated in vitro in the absence of the agent that alters human calcium-activated potassium channels hSK₄ and in the presence of K⁺ or absence of K⁺,
(b) an ADHD control ratio of a mean membrane potential of bipolar control human cells known to have said ADHD incubated in vitro in the presence of the agent that alters human calcium-activated potassium channels hSK₄ and in the absence of K⁺, to a mean membrane potential of the ADHD control human cells incubated in vitro in the absence of the agent that alters human calcium-activated potassium channels hSK₄ and in the presence of K⁺ or absence of K⁺;
   determining the dosage of the at least one drug in the combination drug treatment therapy is an optimal dosage for treating ADHD in the combination therapy when the ratio of the mean membrane potential is not significantly different from the control ratio of (a), is decreased towards the control ratio (a) in comparison to or relative to the ADHD control ratio of (b), and/or is significantly lower in comparison to or relative to the ADHD ratio of (b).

The method may further include obtaining an initial ratio of a mean membrane potential from an initial population of cells from the human patient before the obtaining step.

The method optionally further include modifying the dosage of the at least one drug in the drug therapy treatment for ADHD when the dosage of the at least one drug in the combination therapy is determined to not be the optimal dosage for treating ADHD based on the mean membrane potential.

The agent that may be used include, but is not limited to, a calcium-calmodulin (Ca²⁺/CaM) kinase inhibitor, a diacylglycerol kinase inhibitor, and a PKC inhibitor. Preferably, the agent is a calcium-calmodulin (Ca²⁺/CaM) kinase inhibitor, such as autocamtide-2-related inhibitory peptide (AIP). In another preferred embodiment, the agent is a diacylglycerol kinase inhibitor such as 6-[2-[4-[(4-fluorophenyl)phenylmethylene]-1-piperidinyl]ethyl]-7-methyl-5H-thiazolo[3,2-a]pyrimidin-5-one (ALX).

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

Preferably, the effective amount of methylphenidate is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of methylphenidate.

The human cells that may be used in the present method include, but are not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise methylphenidate and at least one adjunctive agent.

The at least one adjunctive agent used in the method may include, but is not limited to, an anticholinergic agent as described herein.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

Preferably, the effective amount of methylphenidate is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of methylphenidate.

The mean membrane potential test may further include incubating the cells in vitro in buffer comprising a potential-sensitive dye, resuspending the cells in potential-sensitive dye free-buffer, and measuring the cell fluorescence.

### Tenth Embodiment

The present invention further provides a method for monitoring the efficacy of a combination drug treatment therapy for the treatment of attention deficit hyperactivity disorder (ADHD), said method comprising:

obtaining a ratio of a mean membrane potential that is a mean membrane potential of a first population of cells from the ADHD patient incubated in vitro in the presence of an agent that alters human calcium-activated potassium channels (hSK₄) activity and in the absence of K⁺, to a mean membrane potential of a second population of the human patient cells incubated in vitro in the absence of the test agent that alters human calcium-activated potassium channels (hSK₄) activity and the presence of K⁺ or absence of K⁺;

comparing the test ratio to (a) and/or (b):
(a) a control ratio of a mean membrane potential of control human cells known to not have said ADHD incubated in vitro in the presence of the agent that alters human calcium-activated potassium channels hSK₄ and in the absence of K⁺, to a mean membrane potential of the control human cells incubated in vitro in the absence of the agent that alters human calcium-activated potassium channels hSK₄ and in the presence of K⁺ or absence of K⁺,
(b) an ADHD control ratio of a mean membrane potential of bipolar control human cells known to have said ADHD incubated in vitro in the presence of the agent that alters human calcium-activated potassium channels hSK₄ and in the absence of K⁺, to a mean membrane potential of the ADHD control human cells incubated in vitro in the absence of the agent that alters human calcium-activated potassium channels hSK₄ and in the presence of K⁺ or absence of K⁺;

determining the combination drug treatment therapy is efficacious based on the mean membrane potential when the ratio of the mean membrane potential obtained is not significantly different from the control ratio of (a), is decreased towards the control ratio (a) in comparison to or relative to the ADHD control ratio of (b), and/or is significantly lower in comparison to or relative to the bipolar ratio of (b).

The method may further include obtaining an initial ratio of a mean membrane potential from an initial population of cells from the human patient before the obtaining step.

The method optionally further include determining the combination drug treatment therapy is not efficacious based on the mean membrane potential when the ratio of the mean membrane potential obtained is determined to not be efficacious based on the mean membrane potential. Such as when the ratio of the mean membrane potential obtained is higher in comparison to or relative to the control ratio of (a), is increased towards the ADHD control ratio of (b) in comparison to or relative to the control ratio (a), and/or is not significantly different from the ADHD control ratio of (b).

The method may optionally further include adjusting a dosage of one or more agents in the combination drug treatment therapy when the combination therapy is determined to not be efficacious based on the mean membrane potential.

The agent that may be used include, but is not limited to, a calcium-calmodulin (Ca²⁺/CaM) kinase inhibitor, a diacylglycerol kinase inhibitor, and a PKC inhibitor. Preferably, the agent is a calcium-calmodulin (Ca²⁺/CaM) kinase inhibitor, such as autocamtide-2-related inhibitory peptide (AIP). In another preferred embodiment, the agent is a diacylglycerol kinase inhibitor such as 6-[2-[4-[(4-fluorophenyl)phenylmethylene]-1-piperidinyl]ethyl]-7-methyl-5H-thiazolo[3,2-a]pyrimidin-5-one (ALX).

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

Preferably, the effective amount of methylphenidate is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of methylphenidate.

The human cells that may be used in the present method include, but are not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise methylphenidate and at least one adjunctive agent.

The at least one adjunctive agent used in the method may include, but is not limited to, an anticholinergic agent as described herein.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

Preferably, the effective amount of methylphenidate is a dose amount that is less than a dosage of methylphenidate required to provide a therapeutic effect for ADHD therapy when used alone, or is a dose amount that is less than a dosage of methylphenidate required to provide a therapeutically efficacious plasma methylphenidate level for ADHD therapy when used alone. For instance, the effective dose may be a dose that brings the diagnostic probability to the negative range.

Preferably, the effective amount of methylphenidate is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of methylphenidate.

The mean membrane potential test may further include incubating the cells in vitro in buffer comprising a potential-sensitive dye, resuspending the cells in potential-sensitive dye free-buffer, and measuring the cell fluorescence.

### Eleventh Embodiment

In some embodiments thereof, the method includes the steps of:
treating the ADHD patient with a dosage of a combination therapy for treating ADHD;
obtaining at least one sample from the patient which is collected after the treating step;
performing on each sample, a mean membrane potential test including obtaining a ratio of a mean membrane potential from a first population of cells from the sample incubated in vitro in the presence of a compound that alters Na⁺K⁺ ATPase activity and in the absence of K⁺, to a mean membrane potential from a second population of cells from the sample incubated in vitro in the absence of the compound that alters Na⁺K+ ATPase activity and in the presence or absence of K⁺,
comparing the ratio of the mean membrane potential to (a) and/or (b) wherein (a) is a control ratio of a mean membrane potential of control human cells known to not have ADHD incubated in vitro in the presence of the compound that alters Na⁺K⁺ ATPase activity and in the absence of K⁺, to a mean membrane potential of the control human cells incubated in vitro in the absence of the compound that alters Na⁺K⁺ ATPase activity and in the presence or absence of K⁺, and (b) is an ADHD control ratio of a mean membrane potential of ADHD control human cells known to have ADHD incubated in vitro in the presence of the compound that alters Na⁺K⁺ ATPase activity and in the absence of K⁺, to a mean membrane potential of the ADHD control human cells incubated in vitro in the presence of the compound that alters Na⁺K⁺ ATPase activity and in the presence or absence of K⁺;
modifying the drug dosage based on the mean membrane potential test; and
identifying an optimal drug dosage for treating the human patient when the ratio of the mean membrane potential obtained is not significantly different from the control ratio of (a), is decreased towards the control ratio (a) in comparison to or relative to the ADHD control ratio (b), and/or is significantly higher in comparison to or relative to the ADHD control ratio in (b).

The ratio of the mean membrane potential obtained may be not significantly different from or relative to the control ratio of (a), significantly decreased towards the control ratio (a) in comparison to or relative to the ADHD control ratio (b), and/or is significantly lower in comparison to or relative to the ADHF control ratio in (b).

When used with the combination therapies of the present invention, these methods for determining the optimum dose can be used to even further reduce the possibility of side effects.

In other aspects, the present invention relates to monitoring the efficacy of a combination therapy for the treatment of ADHD, by analyzing the membrane potential of cells isolated from a ADHD patient treated with the combination therapy, and calculating a membrane potential ratio therefrom. In some embodiments thereof, the method includes the steps of:
treating the ADHD patient with a dosage of a combination therapy for treating BD;
obtaining at least one sample from the patient which is collected after the treating step;
performing on each sample, a mean membrane potential test including obtaining a ratio of a mean membrane potential from a first population of cells from the sample incubated in vitro in the presence of a compound that alters Na⁺K⁺ ATPase activity and in the absence of K⁺, to a mean membrane potential from a second population of cells from the sample incubated in vitro in the absence of the compound that alters Na⁺K⁺ ATPase activity and in the presence or absence of K⁺,
comparing the ratio of the mean membrane potential to (a) and/or (b) wherein (a) is a control ratio of a mean membrane potential of control human cells known to not have ADHD incubated in vitro in the presence of the compound that alters Na⁺K⁺ ATPase activity and in the absence of K⁺, to a mean membrane potential of the control human cells incubated in vitro in the absence of the compound that alters Na⁺K+ ATPase activity and in the presence or absence of K⁺, and (b) is an ADHD control ratio of a mean membrane potential of ADHD control human cells known to have ADHD incubated in vitro in the presence of the compound that alters Na⁺K⁺ ATPase activity and in the absence of K⁺, to a mean membrane potential of the ADHD control human cells incubated in vitro in the presence of the compound that alters Na⁺K⁺ ATPase activity and in the presence or absence of K;
determining whether the drug dosage is efficacious based on the mean membrane potential test; and
optionally, adjusting the dosage of one or more agents in the combination therapy when the ratio of the mean membrane potential obtained is significantly higher in comparison to or relative to the control ratio of (a) and/or is not different from or relative to the ADHD control ratio of (b).

When used with the combination therapies of the present invention, these monitoring methods can be used to maintain efficacy, while reducing the possibility of side effects.

In some embodiments, the methods of the present invention further include obtaining an initial ratio of a mean membrane potential from an initial population of cells from the BD patient before the treatment step.

The phorbol ester according to the present invention include phorbol 12-myristate 13-acetate (PMA), 12-O-tetradecanoylphorbol 13-acetate, phorbol 12-myristate 13-acetate 4-O-methyl ether, phorbol 12,13-dibutyrate (PDBu), phorbol 12,13-didecanoate (PDD), and phorbol 12,13-dinonanoate 20-homovanillate.

In another embodiment, a compound that decreases the density and/or activity of the potassium channel may be used in the therapy optimization and monitoring methods according to the present invention. For example, low concentrations of ouabain may be useful in determining the effect of the ADHD treatment using MPR™.

Potassium-containing buffers that may be used in the therapy optimization and monitoring methods according to the present invention can be created by adding potassium to the buffers shown in the table above that do not contain potassium. Potassium-containing buffers useful in the methods according to the present invention preferably have a K⁺ concentration in the range of approximately 2 mM to 7 mM, more preferably have a K⁺ concentration of approximately 5 mM, and still more preferably have a K⁺ concentration of 5 mM.

The K⁺-containing buffer may be, for example, a HEPES buffer to which potassium has also been added (5 mMKCl, 4 mMNaHCO₃, 5 mMHEPES, 134 mMNaCl, 2.3 mMCaCl₂, and 5 mM glucose; pH 7.3-7.5, preferably 7.4), and which may be referred to as “regular” or “stock” or “reference” buffer. The K⁺-free buffer used in the examples is a HEPESbuffer without potassium (4 mMNaHCO₃, 5 mMHEPES, 134 mMNaCl, 2.3 mMCaCl₂, and 5 mM glucose. pH 6.6-7.0, preferably 6.8), and is also referred to as “test” buffer.

The membrane potential of a ADHD patient's cells, for the therapy optimization and monitoring methods according to the present invention, may also be ascertained, or confirmed, by any conventional method, such as by examining the fluorescence intensity of a potential-sensitive lipophilic fluorescent dye. The membrane potential is directly proportional to the intensity of fluorescence according to the following equation: I=CV, wherein I is the fluorescence intensity of a lipophilic fluorescent dye, V is the voltage or membrane potential, and C is a constant that can vary depending on a number of factors such as, but not limited to, temperature, lamp intensity, number of cells, concentration of the fluorescent dye, incubation time, and lipid composition of cells used. The calibration and determination of the value for C can be a cumbersome and unreliable procedure. Thus, in some embodiments, by using the ratio of the fluorescence intensity (I₁) of one sample of cells to the fluorescence intensity (I₂) of another sample of cells, the constant (C) is canceled out. Such ratio-metric measurements are preferred over absolute measurements.

Examples of potential-sensitive dyes that may be adapted for use in the present invention, along with their charges and optical responses, are shown below in Table 3 (all available from Molecular Probes Inc., Eugene, Oreg., US).

TABLE 1

| | Structure | |
|---|---|---|
| Dye | (Charge) | Optical Response |
| DiOC₂(3) | Carbocyanine | Slow; fluorescence response to depolarization |
| DiOC₅(3) | (cationic) | depends on staining concentration and detection |
| DiOC₆(3) | | method. |
| DiSC₃(5) | | |
| DiIC₁(5) | | |
| JC-1 | Carbocyanine | Slow; fluorescence emission ratio 582/520 nm |
| JC-9 | (cationic) | increases upon membrane hyperpolarization. |
| Tetramethyl-rhodamine | Rhodamine | Slow; used to obtain unbiased images of |
| methyl and ethyl esters | (cationic) | potential-dependent dye distribution. |
| Rhodamine 123 | | |
| Oxonol V | Oxonol (anionic) | Slow; fluorescence decreases upon membrane |
| Oxonol VI | | hyperpolarization. |
| DiBAC₄(3) | Oxonol (anionic) | Slow; fluorescence decreases upon membrane |
| DiBAC₄(5) | | hyperpolarization. |
| DiSBAC₂(3) | | |
| Merocyanine 540 | Merocyanine | Fast/Slow (biphasic response). |

Indo- (DiI), thia- (DiS) and oxa- (DiO) carbocyanines with short alkyl tails (<7 carbon atoms) were among the first potentiometric fluorescent probes developed. These cationic dyes accumulate on hyperpolarized membranes and are translocated into the lipid bilayer. DiOC₆(3)(3,3′-dihexyloxacarbocyanine iodide), a cell-permeant, voltage sensitive, green-fluorescent dye, has been the most widely used carbocyanine dye for membrane potential measurements, followed closely by DiOC₅(3)(3,3′-dipentylloxacarbocyanine iodide). Thus, in a preferred embodiment of the methods according to the present invention, membrane potentials may be measured using DiOC₆(3) in conjunction with a fluorescence spectrometer.

In one embodiment, the cells are incubated in the presence of K⁺. In another embodiment, the cells are incubated in the absence of K. As used herein, “presence of K⁺” preferably means a K⁺ concentration in the range of approximately 2 mM to 7 mM, preferably approximately 5 mM.

The therapy optimization and monitoring methods according to the present invention may be used with any cell type, such as, but not limited to, erythrocytes, platelets, leukocytes, macrophages, monocytes, dendritic cells, fibroblasts, epidermal cells, mucosal tissue cells, cells in the cerebrospinal fluid, and hair cells. Cells present in blood, skin cells, hair cells, or mucosal tissue cells may be more convenient to use because of the ease of harvesting these cell types.

### Twelfth Embodiment

In a twelfth embodiment, the present invention provides a pharmaceutical combination comprising methylphenidate and at cast one adjunctive agent, as well as a pharmaceutical composition comprising methylphenidate and at least one adjunctive agent; and a pharmaceutically acceptable carrier.

The effective amount of methylphenidate of the pharmaceutical combination or composition may be a dose amount that is less than a dosage of methylphenidate required to provide a therapeutically efficacious plasma methylphenidate level for ADSHD therapy when used alone.

The at least one adjunctive agent of the pharmaceutical combination or composition may be administered at a dose that is less than a dosage of the at least one adjunctive agent required to provide a therapeutically efficacious plasma level of the at least one adjunctive agent when administered alone.

The at least one adjunctive agent of the pharmaceutical combination or composition may include, but is not limited to, an anticholinergic agent as described herein.

Preferably, the at least one adjunctive agent of the pharmaceutical combination or composition is an anticholinergic agent such as an antimuscarinic agent or an antinicotinic agent.

The antimuscarinic agent may include, but is not limited to, trihexyphenidyl, benztropine mesylate, ipratropium, tiotropium, orphenadrine, atropine, flavoxate, oxybutynin, scopolamine, hyoscyamine, tolterodine, fesoterodine, solifenacin, darifenacin, propantheline, biperiden, chlorpheniramine, dicyclomine, dimenhydramine, doxepin, doxylamine, glycopyrrolate, orphenadrine, oxitropium, tropicamide, and pharmaceutically acceptable salts thereof. The antimuscarinic agent may also be selected from a tricyclic antidepressant including butriptyline, clomipramine, imipramine, trimipramine, desipramine, dibenzepin, lofepramine, maprotiline, nortriptyline, protriptyline, amitriptyline, amitriptylinoxide, amoxapine, demexiptiline, dimetacrine, dosulepin, doxepin, fluacizine, imipraminoxide, melitracen, metapramine, nitroxazepine, noxiptiline, pipofezine, propizepine, quinupramine, amineptine, iprindole, opipramol, tianeptine, and pharmaceutically acceptable salts thereof.

The antinicotinic agent may include, but is not limited to, bupropion, dextromethorphan, doxacurium, hexamethonium, mecamylamine, tubocurarine, and pharmaceutically acceptable salts thereof.

### Thirteenth Embodiment

The present invention also provides the following kits.

A kit that may include (a) a K-containing HEPES reference buffer; (b) a K⁺-free HEPES buffer; (c) a potential-sensitive dye; and (d) instructions for performing an assay to determine an optimal combination drug treatment therapy for ADHD.

A kit that may include (a) a K⁺-containing HEPES reference buffer; (b) a K⁺-free HEPES buffer; (c) a potential-sensitive dye; and (d) instructions for performing an assay to optimize a combination drug treatment therapy for ADHD.

A kit that may include (a) a K⁺-containing HEPES reference buffer; (b) a K⁺-free HEPES buffer; (c) a potential-sensitive dye; and (d) instructions for performing an assay to determine an optimum dosage of a drug in combination drug treatment therapy for ADHD.

A kit that may include (a) a K-containing HEPES reference buffer; (b) a K⁺-free HEPES buffer; (c) a potential-sensitive dye; and (d) instructions for performing an assay to monitor the efficacy of a combination drug treatment therapy for ADSHD.

### EXAMPLES

The following examples are provided for illustrative purposes only and are in no way intended to limit the scope of the invention.

### Example 1: Administering Carbachol with Lithium Reduces the Dose of Lithium Needed to be Therapeutic

Carbachol, a choline carbamate, is a cholinergic agonist. At present, carbachol is primarily used in the form of an ophthalmic solution for treating various ophthalmic conditions, such as glaucoma; or for use during ophthalmic surgery. Using the MPR™ test assay described previously by Thiruvengadam (U.S. Pat. No. 7,425,410, incorporated by reference herein in its entirety), the effect of carbachol in combination with lithium on the MPR™ was determined. As mentioned herein, MPR™ is the ratio between the membrane potential (MP) in the test buffer and that in the reference buffer. In these experiments, the reference buffer contained NaCl, CaCl₂), glucose and HEPES, whereas the test buffer contained ethyl alcohol (EtOH) in addition to NaCl, CaCl₂, glucose and HEPES. Lithium, inositol and carbachol were added to the test buffer in these experiments.

Whole blood samples were obtained from RD patients, and a portion from each blood sample was suspended in the test buffer for 20 minutes, and a portion from each blood sample was suspended in the reference buffer for 20 minutes. After this incubation, the samples were centrifuged for five minutes, drained, then re-suspended in their respective buffer (test or reference buffer). These samples were then distributed in 96 well plates, and tested in a plate reader (FLx 800 manufactured by BioTek).

As shown in FIG. 1, the MPR™ value for 1 mM Li was 0.814. However, when 0.5 mM Li, 2.5 μM inositol and 10 μM carbachol were used, the MPR™ value improved to 0.860. (Carbachol is not a psychiatric drug although it is used for the eye. https://www.drugs.com/dosage/carbachol-ophthalmic.html)(Applies to the following strength(s): 0.01%0.75%1.5%2.25%3%). (Instill no more than 0.5 mL into the anterior chamber of the affected eye(s) for the production of miosis during ocular surgery.) Thus, this experiment showed that the MPR™ value obtained with lithium alone, at a concentration of 1 mM, can be significantly improved even at half the dose of lithium (0.5 mM Li), when it is used in combination with what would otherwise be a sub-therapeutic dose of carbachol. This demonstrates the synergistic effect obtained with the combination of lithium and carbachol.

### Example 2: Administering Clozapine with Lithium Reduces the Dose of Lithium Needed to be Therapeutic

Clozapine was discovered in the 1960s, and is a dibenzodiazepine used in mental healthcare. It was the first atypical antipsychotic. Clozapine is also a cholinergic agonist. Clozapine has been used to treat BD (Calabrese et al. “Clozapine for Bipolar Disorder, Letter to the Editor,” Am. J. Psychiatry, 2000, 157: 9; Calabrese et al. “Clozapine for treatment-refractory mania,” Am. J. Psychiatry, 1996, 153: 759-764; Frye et al. “Clozapine in Bipolar Disorder: Treatment Implications for Atypical Antipsychotics,” J. Affec. Disord., 1998, 48: 91-104; and Vangala et al. “Clozapine Associated with Decreased Suicidality in Bipolar Disorder: A Case Report,” Bipolar Disord., 1999.2: 123-124).

However, the side effects of clozapine are significant at presently used therapeutic levels (ranging from 200-1000 ng/ml of blood plasma, see Freudenreich et al. “Clozapine Drug Levels Guide Dosing,” Current Psychiatry, 2009, 8(3)). Using the MPR™ test assay, the effect of clozapine in combination with lithium on de MPR™ was determined. In these experiments, the reference buffer contained NaCl, CaCl₂, glucose and HEPES, whereas the test buffer contained ethyl alcohol (EtOH) in addition to NaCl, CaCl₂, glucose and HEPES. Lithium, inositol and clozapine were added to the test buffer in these experiments.

Whole blood samples were obtained from BD patients, and a portion from each blood sample was suspended in the test buffer for 20 minutes, and a portion from each blood sample was suspended in the reference buffer for 20 minutes. After this incubation, the samples were centrifuged for five minutes, drained, then re-suspended in their respective buffer (test or reference buffer). These samples were then distributed in 96 well plates, and tested in a plate reader (FLx 800 manufactured by BioTek). The results are depicted in FIG. 2.

As shown in FIG. 2, the MPR™ value for 1 mM Li was 0.757. However, when 0.5 mM Li, 2.5 μM inositol and 100 ng/ml clozapine were used, the MPR™ value improved to 0.804. Thus, this experiment showed that the MPR™ value obtained with lithium alone, at a concentration of 1 mM, can be significantly improved even at half the dose of lithium (0.5 mM Li), when it is used in combination with what would otherwise be a sub-therapeutic dose of clozapine. This demonstrates the synergistic effect obtained with the combination of lithium and clozapine.

### Example 3: Administering Donepezil with Lithium Reduces the Dose of Lithium Needed to be Therapeutic

Donepezil is used to improve the cognition and behavior of patients with Alzheimer's disease. Donepezil is a centrally-acting reversible acetylcholinesterase inhibitor. The therapeutic reference range for donepezil is 30-75 ng/ml, see Hefner et al. (“Monitoring (TDM) of donepezil in patients with Alzheimer's dementia,” Pharmacopsychiatry, 2013, 46: A42).

Using the MPR™ test assay, the effect of donepezil in combination with lithium on the MPR™ was determined. In these experiments, the reference buffer contained NaCl, CaCl₂, glucose and HEPES, whereas the test buffer contained ethyl alcohol (EtOH) in addition to NaCl, CaCl₂, glucose and HEPES. Lithium, inositol and donepezil were added to the test buffer in these experiments.

Whole blood samples were obtained from BD patients, and a portion from each blood sample was suspended in the test buffer for 20 minutes, and a portion from each blood sample was suspended in the reference buffer for 20 minutes. After this incubation, the samples were centrifuged for five minutes, drained, then re-suspended in their respective buffer (test or reference buffer). These samples were then distributed in 96 well plates, and tested in a plate reader (FLx 800 manufactured by BioTek). The results are depicted in FIG. 2.

As shown in FIG. 3, the MPR™ value for 1 mM Li was 0.780. However, when 0.5 mM Li, 2.5 μM inositol and 10 ng/ml donepezil were used, the MPR™ value improved to 0.796. Thus, this experiment showed that the MPR™ value obtained with lithium alone, at a concentration of 1 mM, can be significantly improved even at half the dose of lithium (0.5 mM Li), when it is used in combination with what would otherwise be a sub-therapeutic dose of donepezil (10 ng/ml, as compared to the therapeutic reference range of 30-75 ng/ml). This demonstrates the synergistic effect obtained with the combination of lithium and donepezil.

### Example 4

An ADHD patient is tested with a pharmaceutical combination containing 5 mg of MPH and 10 mg of Imipramine (a well known anticholinergic agent). The results show that the MPR values before the combination treatment and after the combination treatment. This result demonstrates that the MPR level is reduced by the combination treatment.

All references, including publications, patent applications, and patents cited herein are hereby incorporated by reference to the same extent as if each reference were individually and specifically indicated to be incorporated by reference and were set forth in its entirety herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### REFERENCES

1. Cade J F J. Lithium salts in the treatment of psychotic excitement. Medical Journal of Australia, 1949, 2: 349-352.
2. Goodwin F K and Jamison K R. Manic-Depressive Illness. Oxford University Press 2007. See also Goodwin F K, Ghaemi N S The impact of the discovery of lithium on psychiatric thought and practice in the USA and Europe. Australian and New Zealand Journal of Psychiatry, 1999, 33: S54-S64.
3. Manji H K, Bowden C L and Belmaker R H. (Ed). Bipolar Medications-Mechanisms of Action, American psychiatric Press, Washington D.C. 2000.
4. Schou M. The early European lithium studies. Australian and New Zealand Journal of Psychiatry, 1999, 33: 539-47.

S. Hokin L E. Lithium increases accumulation of second messenger inositol 1,4,5-triphosphate in brain cortex slices in species ranging from mouse to monkey. Advanced Enzyme Regul. Pergamon Press Ltd. 1993, 33: 299-312.
6. el Mallakh R S. Li R. Worth C A. Peiper S C. Leukocyte transmembrane potential in bipolar illness. J. Affect. Disord. 1996.41: 33-37.
7. Thiruvengadam A. Effect of lithium and sodium valproate ions on resting membrane potentials in neurons: an hypothesis. J. Affect. Disord. 2001. 65, 95-99.
8. Thiruvengadam, A., 2004. The Recent Studies On The Electrobiochemical Aspects Of Bipolar Disorder. In: Brown, M. R. (Ed.). Focus on Bipolar Disorder Research. Nova Science Publishers, New York, pp. 15-35.
9. Thiruvengadam A P, Chandrasekaran K. Evaluating the validity of blood-based membrane potential changes for the identification of bipolar disorder I. J Affect Disord. 2007. 100(1-3):75-82
10. Woodruff D B. el Mallakh R S. Thiruvengadam A P. Validation of a diagnostic screening blood test for bipolar disorder. Annals of Clinical Pychiatry. 2012. 24(2):135-139 11 Newton, A. C., Protein Kinase C: Poised to signal, Am J Physiol Endocrinol Metab298:E395-E402, 2010
12. Nishizuka Y, The role of protein kinase C in cell surface signal transduction and tumour promotion. Nature. 1984 Apr. 19-25; 308(5961):693-8.
13. Dixon J F, Hokin L E, Kinetic analysis of the formation of inositol 1,2-cyclic phosphate in carbachol-stimulated pancreatic minilobules. Half is formed by direct phosphodiesteratic cleavage of phosphatidylinositol, J. Biol. Chem. 1989, 264,11721-11724.
14. Berridge M J, Irvine, inositol phosphates and cell signalling, Nature 341, 197-205 (1989).
15. Stubbs E B, Agranoff B W, Lithium Enhances Muscarinic Receptor-Stimulated CDP-Diacylglycerol Formation in Inositol-Depleted SK-N-SH Neuroblastoma Cells, 1993 JNeurochetn. Vol. 60, No. 4 1292-1299.
16. Calabrese J R, Gajwani P Lamotrigine and Clozapine for Bipolar Disorder, Letter to the Editor, Am J Psychiatry 157:9, 2000, p 1523. (See also) Calabrese J R, Kimmel S E, Woyshvile M J, Rapport D J, Faust C J, Thompson P A, Meltzer H Y: Clozapine for treatment-refractory mania, Am J Psychiatry 1996; 153:759-764
17. Freudenreich O, Clozapine Drug Levels Guide Dosing. 2009, Current Psychiatry, vol 8, No 3.
18. Hefner G, Brueckner A, Geschke K, C Hiemke C, Fellgiebel A, Therapeutic Drug Monitoring (TDM) of donepezil in patients with Alzheimers dementia, Pharmacopsychiatry, 2013; 46-A42.
19. Fieve R R, Lithium Therapy at the Millennium: A Revolutionary Drug Used for 50 Years Faces Competing Options and Possible Demise, Editorial, Bipolar Disorder, 1999, 2: 67-70
20. Frye M A, Ketter T A, Altshuler L L, Denicoff K D, Dunn R T, Kimbrell T A, Cora-Locatelli G, Post R M. Clozapine in Bipolar Disorder: Treatment Implications for Atypical Antipsychotics, 3 Affec. Disord. 1998, 48: 91-104. See also Vangala V R, Brown E S, Suppes T. Clozapine Associated with Decreased Suicidality in Bipolar Disorder: A Case Report. Bipolar Disord 1999, 2: 123-124
21. Kaplan H I, Sadock B J. Synopsis of Psychiatry. 8th Ed. Baltimore: Williams & Wilkins 1988:103-104
22. S C Pandey, J M Davis, D W Schwertz and G N Pandey, Effect of antidepressants and neuroleptics on phosphoinositide metabolism in human platelets, Journal of Pharmacology and Experimental Therapeutics March 1991, 256 (3) 1010-1018.
23. Jania L. Quintero, Maria Isabel Arenas and David E. Garcia, The antidepressant imipramine inhibits M current by activating a phosphatidylinositol 4,5-bisphosphate (PIP2)-dependent pathway in rat sympathetic neurons, British Journal of Pharmacology (2005) 145, 837-843.
24. Ishimatsu et al., Effects of Methylphenidate on the Membrane Potential and Current in Neurons of the Rat Locus Coeruleus, J. Neurophysiol., 87: 1206-1212 (2002).
25. Turbay et al., Methylphenidate Increases cGMP Levels in Rat Brain, Bulletin of Clin. Psychopharmacol., 15(1): 1-4 (2005).

1. A method of determining an optimal combination drug treatment therapy for a patient with attention deficit hyperactivity disorder (ADHD), comprising:
obtaining a ratio of a mean membrane potential that is a mean membrane potential of a first population of cells from the ADHD patient incubated in vitro in the presence of an agent that alters diacylglycerol signaling and in the absence of K+, to a mean membrane potential of a second population of cells from the ADHD patient incubated in vitro in the absence of the test agent that alters diacylglycerol signaling and in the presence of K+ or absence of K+;
comparing the ratio of the mean membrane potential to (a) and/or (b):
(a) a control ratio of a mean membrane potential of first population of control human cells known to not have ADHD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+,
(b) an ADHD control ratio of a mean membrane potential of first population of bipolar control human cells known to have ADHD incubated in vitro in the presence of the agent that alters diacylglycerol signaling and in the absence of K+, to a mean membrane potential of a second population of the ADHD control human cells incubated in vitro in the absence of the agent that alters diacylglycerol signaling and in the presence of K+ or absence of K+;
identifying the optimal combination drug treatment therapy when the ratio of the mean membrane potential obtained is not significantly different from the control ratio in (a), is decreased towards the control ratio in comparison to the ADHD control ratio of (b), and/or is significantly lower than the ADHD control ratio in (b).

2. A method of optimizing a combination drug treatment therapy for a patient with attention deficit hyperactivity disorder (ADHD), comprising the steps of:
obtaining at least one sample from a ADHD patient in a drug therapy treatment for ADHD;
performing on each sample, a mean membrane potential test comprising:
obtaining a ratio of a mean membrane potential that is a mean membrane potential of a first population of cells from the sample incubated in vitro in the presence of an agent that alters diacylglycerol signaling and in the absence of K+, to a mean membrane potential of a second population of the sample incubated in vitro in the absence of the test agent that alters diacylglycerol signaling and in the presence of K+ or absence of K+;
comparing the ratio of the mean membrane potential to (a) and/or (b):
(a) a control ratio of a mean membrane potential of a first population of control human cells known to not have ADHD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+,
(b) an ADHD control ratio of a mean membrane potential of a first population of ADHD control human cells known to have ADHD incubated in vitro in the presence of the agent that alters diacylglycerol signaling and in the absence of K+, to a mean membrane potential of a second population of the ADHD control human cells incubated in vitro in the absence of the agent that alters diacylglycerol signaling and in the presence of K+ or absence of K+;
determining an optimal drug therapy treatment for the ADHD patient based on the mean membrane potential test when the ratio of the mean membrane potential obtained is not significantly different from the control ratio of (a), is decreased towards the control ratio in comparison to the ADHD control ratio of (b), and/or is significantly lower than the ADHD control ratio of (b); and
optionally, modifying at least one drug in the drug therapy treatment for ADHD when the least one drug treatment therapy for ADHD is determined to not be the optimal drug therapy treatment based on the mean membrane potential test.

3. A method for determining an optimum dosage of a drug in a combination drug treatment therapy for the treatment of attention deficit hyperactivity disorder (ADHD), said method comprising:
obtaining at least one sample from a ADHD patient treated with a dosage of a drug in a combination therapy;
performing on each sample, a mean membrane potential test comprising:
obtaining a ratio of a mean membrane potential that is a mean membrane potential of a first population of cells from the ADHD patient incubated in vitro in the presence of an agent that alters diacylglycerol signaling and in the absence of K+, to a mean membrane potential of a second population of cells from the ADHD patient incubated in vitro in the absence of the test agent that alters diacylglycerol signaling and in the presence of K+ or absence of K+;
comparing the ratio of the mean membrane potential to (a) and/or (b):
(a) a control ratio of a mean membrane potential of a first population of cells from a control human known to not have said ADHD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+,
(b) an ADHD control ratio of a mean membrane potential of a first population of cells from a ADHD control human known to have said ADHD incubated in vitro in the presence of the agent that alters diacylglycerol signaling and in the absence of K+, to a mean membrane potential of a second population of cells from the ADHD control human incubated in vitro in the absence of the agent that alters diacylglycerol signaling and in the presence of K+ or absence of K+;
determining the dosage of the drug in the combination drug treatment therapy is an optimal dosage for treating ADHD in the combination therapy based on the mean membrane potential test when the ratio of the mean membrane potential obtained is not significantly different from the control ratio of (a), is decreased towards the control ratio in comparison to the ADHD control ratio of (b), and/or is significantly lower than the ADHD control ratio of (b); and
optionally, modifying the dosage of the drug in the combination drug treatment therapy when the dosage of the drug in the combination therapy is determined to be not the optimal dosage for treating ADHD based on the mean membrane potential test.

4. A method for monitoring the efficacy of a combination drug treatment therapy for the treatment of attention deficit hyperactivity disorder (ADHD), said method comprising:
obtaining at least one sample from a ADHD patient treated with a combination drug treatment therapy for treating ADHD;
performing on each sample, a mean membrane potential test comprising:
obtaining a ratio of a mean membrane potential that is a mean membrane potential of a first population of cells from the ADHD patient incubated in vitro in the presence of an agent that alters diacylglycerol signaling and in the absence of K+, to a mean membrane potential of a second population of cells from the ADHD patient incubated in vitro in the absence of the test agent that alters diacylglycerol signaling and in the presence of K+ or absence of K+;
comparing the ratio of the mean membrane potential to (a) and/or (b):
(a) a control ratio of a mean membrane potential of a first population of cells from a control human known to not have said ADHD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+,
(b) an ADHD control ratio of a mean membrane potential of a first population of cells from a ADHD control human known to have said ADHD incubated in vitro in the presence of the agent that alters diacylglycerol signaling and in the absence of K+, to a mean membrane potential of a second population of cells from the ADHD control human incubated in vitro in the absence of the agent that alters diacylglycerol signaling and in the presence of K+ or absence of K+;
determining the combination drug treatment therapy is efficacious based on the mean membrane potential test when the ratio of the mean membrane potential obtained is not significantly different from the control ratio in (a), is decreased towards the control ratio in comparison to the ADHD control ratio of (b), and/or is significantly lower than the ADHD control ratio in (b); and
optionally, adjusting a dosage of one or more agents in the combination drug treatment therapy when the combination therapy is determined to be not efficacious based on the mean membrane potential test.

5. The method according to claim 1, further comprising obtaining an initial ratio of a mean membrane potential from an initial population of cells from the human patient before the obtaining step.

6. The method of claim 1, wherein the human cells is selected from the group consisting of red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells.

7. The method of claim 6, wherein the human cells is selected from the group consisting of red blood cells and lymphoblasts.

8. The method of claim 1, wherein the combination drug treatment therapy is synergistic combination.

9. The method of claim 8, wherein the combination drug treatment therapy comprises methylphenidate and at least one adjunctive agent.

10. The method of claim 9, wherein the at least one adjunctive agent is an anticholinergic agent.

11. The method of claim 1, wherein the agent that alters diacylglycerol signaling is selected from the group consisting of a calcium-calmodulin (Ca2+/CaM) kinase inhibitor, a diacylglycerol kinase inhibitor, a protein kinase C inhibitor, and an agent that affects calcium-activated potassium (CaK) channels.

12. The method of claim 11, wherein the agent is a calcium-calmodulin (Ca2+/CaM) kinase inhibitor.

13. The method of claim 12, wherein the calcium-calmodulin (Ca2+/CaM) kinase inhibitor is autocamtide-2-related inhibitory peptide (AIP).

14. The method of claim 11, wherein the agent is a diacylglycerol kinase inhibitor.

15. The method of claim 14, wherein the diacylglycerol kinase inhibitor is 6-[2-[4-[(4-fluorophenyl)phenylmethylene]-1-piperidinyl]ethyl]-7-methyl-5H-thiazolo[3,2-alpyrimidin-5-one (ALX).

16. The method of claim 1, wherein the mean membrane potential test further comprises incubating the cells in vitro in buffer comprising a potential-sensitive dye, resuspending the cells in potential-sensitive dye free-buffer, and measuring the cell fluorescence.

17. The method of claim 1, wherein the agent that alters K+ channel activity is ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof.

18. The method of claim 17, wherein the agent that alters K+ channel activity is ethanol.

19. A method of treating attention deficit hyperactivity disorder (ADHD), comprising administering an effective amount of methylphenidate and at least one adjunctive agent to a human patient with ADHD.

20. A method of increasing the therapeutic efficacy of methylphenidate for the treatment of attention deficit hyperactivity disorder (ADHD), comprising administering an effective amount of methylphenidate with at least one adjunctive agent, to a human patient with ADHD.

21. The method of claim 19, wherein the at least one adjunctive agent and the methylphenidate to form a synergistic combination or composition to treat said ADSHD.

22. The method of claim 19, wherein the effective amount of methylphenidate is a dose amount that is less than a dosage of methylphenidate required to provide a therapeutically efficacious plasma methylphenidate level for ADHD therapy when used alone.

23. The method of claim 19, wherein the at least one adjunctive agent is administered at a dose that is less than a dosage of the at least one adjunctive agent required to provide a therapeutically efficacious plasma level of the at least one adjunctive agent when administered alone.

24. The method of claim 19, wherein the at least one adjunctive agent is an anticholinergic agent.

25. A pharmaceutical combination comprising methylphenidate or pharmaceutically acceptable salt thereof, and at least one adjunctive agent.

26. A pharmaceutical composition comprising methylphenidate or pharmaceutically acceptable salt thereof, and at least one adjunctive agent.

27. The pharmaceutical composition of claim 26, further comprising a pharmaceutically acceptable carrier.

28. The pharmaceutical combination or composition of claim 25, wherein the effective amount of the methylphenidate is a dose amount that is less than a dosage of the methylphenidate required to provide a therapeutically efficacious plasma methylphenidate level for ADHD therapy when used alone.

29. The pharmaceutical combination or composition of claim 25, wherein the at least one adjunctive agent is administered at a dose that is less than a dosage of the at least one adjunctive agent required to provide a therapeutically efficacious plasma level of the at least one adjunctive agent when administered alone.

30. The pharmaceutical combination or composition of claim 25, wherein the at least one adjunctive agent is an anticholinergic agent.

31. A kit comprising:
(a) a reference buffer;
(b) a test buffer;
(c) a potential-sensitive dye; and
(d) instructions for performing an assay to determine an optimal combination drug treatment therapy for attention deficit hyperactivity disorder (ADHD).

32. A kit comprising:
(a) a reference buffer;
(b) a test buffer;
(c) a potential-sensitive dye; and
(d) instructions for performing an assay to optimize a combination drug treatment therapy for attention deficit hyperactivity disorder (ADHD).

33. A kit comprising:
(a) a reference buffer;
(b) a test buffer;
(c) a potential-sensitive dye; and
(d) instructions for performing an assay to determine an optimum dosage of a drug in combination drug treatment therapy for attention deficit hyperactivity disorder (ADHD).

34. A kit comprising:
(a) a reference buffer;
(b) a test buffer;
(c) a potential-sensitive dye; and
(d) instructions for performing an assay to monitor the efficacy of a combination drug treatment therapy for attention deficit hyperactivity disorder (ADHD).

35. The kit of claim 31, wherein the reference buffer contains NaCl, Cacl2, glucose and hepes.

36. The kit of claim 31, wherein the test buffer contains ethyl alcohol, NaCl, Cacl2, glucose and hepes.
